(19) United States
(12) Patent Application Publication
(10) Pub. No.: US 2006/0075520 A1
(43) Pub. Date: Apr. 6, 2006

(54) Brittle stalk 2 polynucleotides, polypeptides and uses thereof

(76) Inventors: Ada S. Ching, Wilmington, DE (US); J. Antoni Rafalski, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/242,507

(22) Filed: Oct. 3, 2005

Related U.S. Application Data
(60) Provisional application No. 60/615,868, filed on Oct. 6, 2004.

(51) Int. Cl. A01H 1/00; A01H 5/00; C12N 5/04; C07H 21/04

(52) U.S. Cl. 800/287; 800/320.1; 435/412; 536/23.6

(57) ABSTRACT

This invention relates to an isolated polynucleotide encoding a BRITTLE STALK 2 (BK2) polypeptide. The invention also relates to the construction of a chimeric gene encoding all or a portion of the BK2 polypeptide, in sense or antisense orientation, wherein expression of the chimeric gene results in production of altered levels of the BK2 polypeptide in a transformed host cell.

This application claims the benefit of U.S. Provisional Application No. 60/615,868, filed Oct. 6, 2004, the entire content of which is herein incorporated by reference.

### FIELD OF THE INVENTION

The field of invention relates to plant molecular biology, and in particular, to BRITTLE STALK 2 genes, BRITTLE STALK 2 polypeptides, and uses thereof.

### BACKGROUND OF THE INVENTION

Plant mechanical strength (brittleness) is one of the most important agronomic traits. Plant mutants that are defective in stem strength have been isolated and characterized. Barley brittle culm (bc) mutants were first described based on the physical properties of the culms, which have an 80% reduction in the amount of cellulose and a twofold decrease in breaking strength compared with those of wildtype plants (Kokubo et al., Plant Physiol. 97:509-514 (1991)). Rice brittle culm1 (bc1) mutants show a reduction in cell wall thickness and cellulose content (Qian et al., Chi. Sci. Bull. 46:2082-2085 (2001)). Li et al. described the identification of rice BRITTLE CULM1 (BC1), a gene that encodes a COBRA-like protein (The Plant Cell 15(9):2020-2031 (2003)). Their findings indicated that BC1 functions in regulating the biosynthesis of secondary cell walls to provide the main mechanical strength for rice plants.

The stalk of maize brittle stalk 2 (bk2) mutants exhibits a dramatically reduced mechanical strength compared to its wild type counterpart (Langham, MNL 14:21-22 (1940)). Maize bk2 mutants have stalk and leaves that are very brittle and break easily. The main chemical constituent deficient in the mutant stalk is cellulose. Therefore, stalk mechanical strength appears to be dependent primarily on the amount of cellulose in a unit length of the stalk below the ear.

As insufficient stalk strength is a major problem in corn breeding. It is desirable to provide compositions and methods for manipulating cellulose concentration in the cell wall and thereby alter plant stalk strength and/or quality for improved standability or silage.

### SUMMARY OF THE INVENTION

The present invention includes:

In a preferred first embodiment, an isolated polynucleotide comprising (a) a nucleic acid sequence encoding a polypeptide having an amino acid sequence of at least 85%, 90%, 95%, 96%, 97%, 98%, 99% or 100% sequence identity, based on the Clustal V method of alignment, when compared to SEQ ID NO:59, wherein expression of said polypeptide in a plant transformed with said isolated polynucleotide results in alteration of the stalk mechanical strength of said transformed plant when compared to a corresponding untransformed plant; or (b) a complement of the nucleotide sequence, wherein the complement and the nucleotide sequence consist of the same number of nucleotides and are 100% complementary. Preferably, expression of said polypeptide results in an increase in the stalk mechanical strength, and even more preferably, the plant is maize.

In a preferred second embodiment, an isolated polynucleotide comprising (a) a nucleic acid sequence encoding a polypeptide having an amino acid sequence of at least 85%, 90%, 95%, 96%, 97%, 98%, 99% or 100% sequence identity, based on the Clustal V method of alignment, when compared to SEQ ID NO:59, wherein expression of said polypeptide in a plant exhibiting a brittle stalk 2 mutant phenotype results in an increase of stalk mechanical strength of said plant; or (b) a complement of the nucleotide sequence, wherein the complement and the nucleotide sequence consist of the same number of nucleotides and are 100% complementary. Preferably, the plant is maize.

In a preferred third embodiment, an isolated polynucleotide comprising (a) a nucleotide sequence encoding a polypeptide associated with stalk mechanical strength, wherein said polypeptide has an amino acid sequence comprising SEQ ID NO:59, or (b) a complement of the nucleotide sequence, wherein the complement and the nucleotide sequence consist of the same number of nucleotides and are 100% complementary.

In a preferred fourth embodiment, a vector comprising a polynucleotide of the present invention.

In a preferred fifth embodiment, a recombinant DNA construct comprising a polynucleotide of the present invention, operably linked to at least one regulatory sequence.

In a preferred six embodiment, a recombinant DNA construct of the present invention, further comprising an enhancer.

In a preferred seventh embodiment, a cell, plant, or seed comprising a recombinant DNA construct of the present invention.

In a preferred eighth embodiment, a method for transforming a cell, comprising transforming a cell with a polynucleotide of the present invention.

In a preferred ninth embodiment, a method for producing a plant comprising transforming a plant cell with a polynucleotide of the present invention, and regenerating a plant from the transformed plant cell.

In a preferred tenth embodiment, a method of altering stalk mechanical strength in a plant, comprising (a) transforming a plant, preferably a maize plant, with a recombinant DNA construct of the present invention; and (b) growing the transformed plant under conditions suitable for the expression of the recombinant DNA construct, said grown transformed plant having an altered level of stalk mechanical strength when compared to a corresponding nontransformed plant. Preferably, the grown transformed plant has an increased level of stalk mechanical strength when compared to a corresponding nontransformed plant.

In a preferred eleventh embodiment, a plant transformed with a recombinant DNA construct of the present invention and having an increased level of stalk mechanical strength when compared to a corresponding nontransformed plant.

In a preferred twelfth embodiment, a method for determining whether a plant exhibits a brittle stalk 2 mutant genotype comprising: (a) isolating genomic DNA from a subject; (b) performing a PCR on the isolated genomic DNA using primer pair AGGGAGCTTGTGCTGCTA (SEQ ID NO:53) and GCAGCTTCACCGTCTTGTT (SEQ ID NO:54); and (c) analyzing results of the PCR for the presence of a larger DNA fragment as an indication that the subject exhibits the brittle stalk 2 mutant genotype.

In a preferred thirteenth embodiment, a transgenic plant whose genome comprises a homozygous disruption of a BRITTLE STALK 2 gene, wherein said disruption comprises an insertion in said gene and results in said transgenic plant exhibiting reduced stalk mechanical strength when compared to its wild type counterpart. Preferably, the disruption comprises the insertion of SEQ ID NO:60.

In a preferred fourteenth embodiment, an isolated polynucleotide comprising SEQ ID NO:61.

### BRIEF DESCRIPTION OF THE FIGURES AND SEQUENCE LISTINGS

The invention can be more fully understood from the following detailed description and the accompanying drawings and Sequence Listing which form a part of this application.

FIGS. 1A-1B show the genotypic scores that were used to map each marker gene relative to Contig 2 (SEQ ID NO:28). The locus represented by Contig 2 (SEQ ID NO:28) was found to lie between markers umc95 and umc1492. A signifies individuals homozygous for the B73 allele, B signifies individuals homozygous for the Mo17 allele and H signifies heterozygous individuals.

FIGS. 2A-2C show an alignment of the amino acid sequence reported herein of a Zea mays BRITTLE STALK 2 polypeptide (SEQ ID NO:59) to the amino acid sequence of an Oryza sativa BRITTLE CULM1 polypeptide(SEQ ID NO:2). The sequences are 84.4% identical using the Clustal V method of alignment.

FIG. 3 shows a schematic of the BK2 transgene construct which directs expression of the BK2 polypeptide in the stalk by operably linking the BK2 cDNA to the alfalfa stalk specific S2A gene promoter (see Example 8).

FIG. 4 shows a schematic of BK2 genomic DNA from the Mo17 wild type maize (SEQ ID NO. 61). Exon 1 is from nucleotide 1 to 158 (with the 5′ UTR from nucleotide 1 to 79), exon 2 is from nucleotide 286 to 1269, exon 3 is from nucleotide 1357 to 1798, the C-terminal region starts at nucleotide 1562, and the stop codon is at nucleotides 1644-1646. Sites in exon 2 where insertions have been found in mutant plants are indicated as “bk2 insertion site” (between nucleotides 292-293) and “TUSC insertion site” (between nucleotides 588-589).

SEQ ID NO:1 is the complete coding sequence of the BRITTLE CULM1 gene from Oryza sativa (japonica cultivar-group) (NCBI General Identifier No. 34014145).

SEQ ID NO:2 is the amino acid sequence of BRITTLE CULM1 from Oryza sativa (japonica cultivar-group) (NCBI General Identifier No. 34014146).

SEQ ID NO:3 is the nucleotide sequence of clone cdr1f.pk006.d4:fis.

SEQ ID NO:4 is the nucleotide sequence of clone cen3n.pk0203.g1a.

SEQ ID NO:5 is the nucleotide sequence of clone cest1s.pk003.o23.

SEQ ID NO:6 is the nucleotide sequence of clone p0018.chsug94r.

SEQ ID NO:7 is the nucleotide sequence of clone p0032.crcau13r.

SEQ ID NO:8 is the nucleotide sequence of clone cbn10.pk0006.f4.

SEQ ID NO:9 is the nucleotide sequence of clone cdt2c.pk003.k7.

SEQ ID NO:10 is the nucleotide sequence of clone cgs1c.pk001.d14a.

SEQ ID NO:11 is the nucleotide sequence of clone cr1n.pk0144.a2a.

SEQ ID NO:12 is the nucleotide sequence of clone cr1n.pk0144.a2b.

SEQ ID NO:13 is the nucleotide sequence of clone csc1c.pk005.k4.

SEQ ID NO:14 is the nucleotide sequence of clone ctst1s.pk008.l15.

SEQ ID NO:15 is the nucleotide sequence of clone ctst1s.pk014.g20.

SEQ ID NO:16 is the nucleotide sequence of clone p0058.chpbr83r.

SEQ ID NO:17 is the nucleotide sequence of clone cdt2c.pk005.i7a.

SEQ ID NO:18 is the nucleotide sequence of clone p0019.clwah76ra.

SEQ ID NO:19 is the nucleotide sequence of TIGR Assembly Number AZM2_{—}14907.

SEQ ID NO:20 is the nucleotide sequence of TIGR Assembly Number AZM2_{—}36996.

SEQ ID NO:21 is the nucleotide sequence of TIGR Assembly Number AZM2_{—}14120.

SEQ ID NO:22 is the nucleotide sequence of TIGR Assembly Number AZM2_{—}33700.

SEQ ID NO:23 is the nucleotide sequence of TIGR Assembly Number OGACO44TC.

SEQ ID NO:24 is the nucleotide sequence of TIGR Assembly Number AZM2_{—}13022.

SEQ ID NO:25 is the nucleotide sequence of TIGR Assembly Number OGAMW81TM.

SEQ ID NO:26 is the nucleotide sequence of TIGR Assembly Number AZM2_{—}37864.

SEQ ID NO:27 (also known as Contig 1) is the nucleotide sequence of the contig derived from clones cdr1f.pk006.d4:fis, cen3n.pk0203.g1a, cest1s.pk003.o23 p0018.chsug94r and p0032.crcau13r.

SEQ ID NO:28 (also known as Contig 2) is the nucleotide sequence of the contig derived from the TIGR GSS sequence AZM2_{—}14907 and clones cbn10.pk0006.f4, cdt2c.pk003.k7, cgs1c.pk001.d14a, cr1n.pk0144.a2a, cr1n.pk0144.a2b, csc1c.pk005.k4, ctst1s.pk008.l15, ctst1s.pk014.g20 and p0058.chpbr83r.

SEQ ID NO:29 (also known as Contig 3) is the nucleotide sequence of the contig derived from clones cdt2c.pk005.i7a and p0019.clwah76ra.

SEQ ID NO:30 is the nucleotide sequence of clone p0102.ceraf50r.

SEQ ID NO:31 is the left primer designed from Contig 1 (SEQ ID NO:27) used to amplify from a set of genomic DNA prepared from the oat-maize addition lines.

SEQ ID NO:32 is the right primer designed from Contig 1 (SEQ ID NO:27) used to amplify from a set of genomic DNA prepared from the oat-maize addition lines.

SEQ ID NO:33 is the left primer designed from Contig 2 (SEQ ID NO:28) used to amplify from a set of genomic DNA prepared from the oat-maize addition lines.

SEQ ID NO:34 is the right primer designed from Contig 2 (SEQ ID NO:28) used to amplify from a set of genomic DNA prepared from the oat-maize addition lines.

SEQ ID NO:35 is the left primer designed from Contig 3 (SEQ ID NO:29) used to amplify from a set of genomic DNA prepared from the oat-maize addition lines.

SEQ ID NO:36 is the right primer designed from Contig 3 (SEQ ID NO:29) used to amplify from a set of genomic DNA prepared from the oat-maize addition lines.

SEQ ID NO:37 is the left primer designed from AZM2_{—}36996 (SEQ ID NO:20) used to amplify from a set of genomic DNA prepared from the oat-maize addition lines.

SEQ ID NO:38 is the right primer designed from AZM2_{—}36996 (SEQ ID NO:20) used to amplify from a set of genomic DNA prepared from the oat-maize addition lines.

SEQ ID NO:39 is the left primer designed from p0102.ceraf50r (SEQ ID NO:30) used to amplify from a set of genomic DNA prepared from the oat-maize addition lines.

SEQ ID NO:40 is the right primer designed from p0102.ceraf50r (SEQ ID NO:30) used to amplify from a set of genomic DNA prepared from the oat-maize addition lines.

SEQ ID NO:41 is the left primer for CAPS marker Contig 2 used in Example 5

SEQ ID NO:42 is the right primer for CAPS marker Contig 2 used in Example 5

SEQ ID NO:43 is the left primer for SSR marker BNLG1375 used in Example 5.

SEQ ID NO:44 is the right primer for SSR marker BNLG1375 used in Example 5.

SEQ ID NO:45 is the left primer for SSR marker UMC95 used in Example 5.

SEQ ID NO:46 is the right primer for SSR marker UMC95 used in Example 5.

SEQ ID NO:47 is the left primer for SSR marker UMC1492 used in Example 5.

SEQ ID NO:48 is the right primer for SSR marker UMC1492 used in Example 5.

SEQ ID NO:49 is the left primer for SSR marker UFG70 used in Example 5.

SEQ ID NO:50 is the right primer for SSR marker UFG70 used in Example 5.

SEQ ID NO:51 is the left primer of primer ps231 designed from Contig 2 (SEQ ID NO:28) used in Example 6.

SEQ ID NO:52 is the right primer of primer ps231 designed from Contig 2 (SEQ ID NO:28) used in Example 6.

SEQ ID NO:53 is the left primer of primer ps238 designed from Contig 2 (SEQ ID NO:28) used in Example 6.

SEQ ID NO:54 is the right primer of primer ps238 designed from Contig 2 (SEQ ID NO:28) used in Example 6.

SEQ ID NO:55 is a primer used to screen the TUSC population in Example 7.

SEQ ID NO:56 is a primer used to screen the TUSC population in Example 7.

SEQ ID NO:57 is the Mutator TIR primer used in Example 7.

SEQ ID NO:58 is the nucleotide sequence comprising the entire cDNA insert in clone csc1c.pk005.k4:fis encoding SEQ ID NO:59.

SEQ ID NO:59 is the deduced amino acid sequence of a corn BRITTLE STALK 2 (BK2) polypeptide derived from the nucleotide sequence set forth in SEQ ID NO:58

SEQ ID NO:60 is the nucleotide sequence of the insertion in a brittle stalk 2 (bk2) mutant.

SEQ ID NO:61 is the genomic DNA sequence of the corn BRITTLE STALK 2 (BK2) gene in Mo17.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219(2):345-373 (1984) which are herein incorporated by reference. The symbols and format used for nucleotide and amino acid sequence data comply with the rules set forth in 37 C.F.R. §1.822. The sequence descriptions and Sequence Listing attached hereto comply with the rules governing nucleotide and/or amino acid sequence disclosures in patent applications as set forth in 37 C.F.R. §1.821-1.825.

### DETAILED DESCRIPTION OF THE INVENTION

All patents, patent applications, and publications cited throughout the application are hereby incorporated by reference in their entirety.

In the context of this disclosure, a number of terms shall be utilized.

The term “BRITTLE STALK 2 (BK2) gene” is a gene of the present invention and refers to a non-heterologous genomic form of a full-length BRITTLE STALK 2 (BK2) polynucleotide. In a preferred embodiment, the BRITTLE STALK 2 gene comprises SEQ ID NO:58 or 61.

The term “BRITTLE STALK 2 (BK2) polypeptide” refers to a polypeptide of the present invention and may comprise one or more amino acid sequences, in glycosylated or non-glycosylated form. A “BRITTLE STALK 2 (BK2) protein” comprises a BRITTLE STALK 2 polypeptide.

The term “amplified” means the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

The term “chromosomal location” includes reference to a length of a chromosome which may be measured by reference to the linear segment of DNA which it comprises. The chromosomal location can be defined by reference to two unique DNA sequences, i.e., markers.

The term “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes in that pair to be followed. A genotype may be defined by use of one or a plurality of markers.

The term “plant” includes reference to whole plants, plant parts or organs (e.g., leaves, stems, roots, etc.), plant cells, seeds and progeny of same. Plant cell, as used herein includes, without limitation, cells obtained from or found in the following: seeds, suspension cultures, embryos, meristematic regions, callus tissue, leaves, roots, shoots, gametophytes, sporophytes, pollen and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. Particularly preferred plants include maize, soybean, sunflower, sorghum, canola, wheat, alfalfa, cotton, rice, barley and millet.

The term “isolated nucleic acid fragment” is used interchangeably with “isolated polynucleotide” and is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of recombinant DNA constructs to produce the desired phenotype in a transformed plant. Recombinant DNA constructs can be designed for use in co-suppression or antisense by linking a nucleic acid fragment or subfragment thereof, whether or not it encodes an active enzyme, in the appropriate orientation relative to a plant promoter sequence.

“Cosuppression” refers to the production of sense RNA transcripts capable of suppressing the expression of identical or substantially similar native genes (U.S. Pat. No. 5,231,020).

“Antisense inhibition” refers to the production of antisense RNA transcripts capable of suppressing the expression of the target protein.

As stated herein, “suppression” refers to the reduction of the level of enzyme activity or protein functionality (e.g., a phenotype associated with a protein, such as stalk mechanical strength associated with polypeptides of the present invention) detectable in a transgenic plant when compared to the level of enzyme activity or protein functionality detectable in a plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to the decrease in translation of the native mRNA into an active enzyme or functional protein. It may also be due to the transcription of the native DNA into decreased amounts of mRNA and/or to rapid degradation of the native mRNA. The term “native enzyme” refers to an enzyme that is produced naturally in the desired cell.

“Gene silencing,” as used herein, is a general term that refers to decreasing mRNA levels as compared to wild-type plants, does not specify mechanism and is inclusive, and not limited to, anti-sense, cosuppression, viral-suppression, hairpin suppression and stem-loop suppression.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. For example, alterations in a nucleic acid fragment which result in the production of a chemically equivalent amino acid at a given site, but do not effect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. It is therefore understood, as those skilled in the art will appreciate, that the invention encompasses more than the specific exemplary sequences.

Moreover, the skilled artisan recognizes that substantially similar nucleic acid sequences encompassed by this invention are also defined by their ability to hybridize, under moderately stringent conditions (for example, 1×SSC, 0.1% SDS, 60° C.) with the sequences exemplified herein, or to any portion of the nucleotide sequences reported herein and which are functionally equivalent to the gene or the promoter of the invention. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

With respect to the degree of substantial similarity between the target (endogenous) mRNA and the RNA region in the construct having homology to the target mRNA, such sequences should be at least 25 nucleotides in length, preferably at least 50 nucleotides in length, more preferably at least 100 nucleotides in length, again more preferably at least 200 nucleotides in length, and most preferably at least 300 nucleotides in length; and should be at least 80% identical, preferably at least 85% identical, more preferably at least 90% identical, and most preferably at least 95% identical.

Sequence alignments and percent similarity calculations may be determined using a variety of comparison methods designed to detect homologous sequences including, but not limited to, the Megalign program of the LASARGENE bioinformatics computing suite (DNASTAR Inc., Madison, Wis.). Unless stated otherwise, multiple alignment of the sequences provided herein were performed using the Clustal method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain a “percent identity” by viewing the “sequence distances” table on the same program.

Unless otherwise stated, “BLAST” sequence identity/similarity values provided herein refer to the value obtained using the BLAST 2.0 suite of programs using default parameters (Altschul et al., Nucleic Acids Res. 25:3389-3402 (1997)). Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=⁻4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

The term “recombinant” means, for example, that a nucleic acid sequence is made by an artificial combination of two otherwise separated segments of sequence, e.g., by chemical synthesis or by the manipulation of isolated nucleic acids by genetic engineering techniques.

As used herein, “contig” refers to a nucleotide sequence that is assembled from two or more constituent nucleotide sequences that share common or overlapping regions of sequence homology. For example, the nucleotide sequences of two or more nucleic acid fragments can be compared and aligned in order to identify common or overlapping sequences. Where common or overlapping sequences exist between two or more nucleic acid fragments, the sequences (and thus their corresponding nucleic acid fragments) can be assembled into a single contiguous nucleotide sequence.

“Codon degeneracy” refers to divergence in the genetic code permitting variation of the nucleotide sequence without effecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein. A gene encompasses regulatory sequences preceding (5′ non-coding sequences) and following (3′ non-coding sequences) the coding sequence.

“Native gene” refers to a gene as found in nature with its own regulatory sequences.

“Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, and arranged in a manner different than that found in nature.

A “foreign” gene refers to a gene not normally found in the host organism, that is introduced into the host organism by gene transfer. Foreign genes can comprise native genes inserted into a non-native organism, or chimeric genes.

A “transgene” is a gene that has been introduced into the genome by a transformation procedure.

An “allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

“Coding sequence” refers to a DNA fragment that codes for a polypeptide having a specific amino acid sequence.

The term “expression”, as used herein, refers to the production of a functional end-product e.g., a mRNA or a protein (precursor or mature).

“Mature” protein refers to a post-translationally processed polypeptide; i.e., one from which any pre- or pro-peptides present in the primary translation product have been removed. “Precursor” protein refers to the primary product of translation of mRNA; i.e., with pre- and pro-peptides still present. Pre- and pro-peptides may be and are not limited to intracellular localization signals.

“RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Sense” RNA refers to RNA transcript that includes the mRNA and can be translated into protein within a cell or in vitro.

“Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Functional RNA” refers to antisense RNA, ribozyme RNA, or other RNA that may not be translated, yet has an effect on cellular processes. The terms “complement” and “reverse complement” are used interchangeably herein with respect to mRNA transcripts, and are meant to define the antisense RNA of the message.

The term “recombinant DNA construct” refers to a DNA construct assembled from nucleic acid fragments obtained from different sources. The types and origins of the nucleic acid fragments may be very diverse.

The term “operably linked” refers to the association of nucleic acid fragments on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

“Regulatory sequences” refer to nucleotides located upstream (5′ non-coding sequences), within, or downstream (3′ non-coding sequences) of a coding sequence, and which influence the transcription, RNA processing, stability, or translation of the associated coding sequence.

“Promoter” refers to a region of DNA capable of controlling the expression of a coding sequence or functional RNA. The promoter sequence consists of proximal and more distal upstream elements. These upstream elements are often referred to as enhancers. An “enhancer” is a DNA sequence that can stimulate promoter activity, and may be an innate element of the promoter or a heterologous element inserted to enhance the level or tissue-specificity of a promoter.

The “translation leader sequence” refers to a polynucleotide fragment located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Mol. Biotechnol. 3:225-236).

An “intron” is an intervening sequence in a gene that does not encode a portion of the protein sequence. Thus, such sequences are transcribed into RNA but are then excised and are not translated. The term is also used for the excised RNA sequences.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989. Transformation methods are well known to those skilled in the art and are described below.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

“Stable transformation” refers to the transfer of a nucleic acid fragment into a genome of a host organism, including nuclear and organellar genomes, resulting in genetically stable inheritance.

In contrast, “transient transformation” refers to the transfer of a nucleic acid fragment into the nucleus, or DNA-containing organelle, of a host organism resulting in gene expression without integration or stable inheritance.

Host organisms containing the transformed nucleic acid fragments are referred to as “transgenic” organisms.

Turning now to preferred embodiments:

In one preferred embodiment of the present invention, an isolated polynucleotide comprises (a) a nucleic acid sequence encoding a polypeptide having an amino acid sequence of at least 85%, 90%, 95%, 96%, 97%, 98%, 99% or 100% sequence identity, based on the Clustal V method of alignment, when compared to SEQ ID NO:59, wherein expression of said polypeptide in a plant transformed with said isolated polynucleotide results in alteration of the stalk mechanical strength of said transformed plant when compared to a corresponding untransformed plant; or (b) a complement of the nucleotide sequence, wherein the complement and the nucleotide sequence consist of the same number of nucleotides and are 100% complementary. Preferably, expression of said polypeptide results in an increase in the stalk mechanical strength, and even more preferably, the plant is maize.

In another preferred embodiment of the present invention, an isolated polynucleotide comprises (a) a nucleic acid sequence encoding a polypeptide having an amino acid sequence of at least 85%, 90%, 95%, 96%, 97%, 98%, 99% or 100% sequence identity, based on the Clustal V method of alignment, when compared to SEQ ID NO:59, wherein expression of said polypeptide in a plant exhibiting a bfittle stalk 2 mutant phenotype results in an increase of stalk mechanical strength of said plant; or (b) a complement of the nucleotide sequence, wherein the complement and the nucleotide sequence consist of the same number of nucleotides and are 100% complementary. Preferably, the plant is maize.

Several methods may be used to measure the stalk mechanical strength of plants. Preferably, the mechanical strength may be measured with an electromechanical test system. In the case of maize stalk mechanical strength, in a preferred method, the internodes below the ear may be subjected to a three-point bend test using an Instron, Model 4411 (Instron Corporation, 100 Royall Street, Canton, Mass. 02021), with a span-width of 200 mm between the anchoring points and a speed of 200 mm/minute of the third point attached to a load cell; the load needed to break the internode can be used as a measure of mechanical strength (hereinafter “the three-point bend test”). Internodal breaking strength has been shown to be highly correlated with the amount of cellulose per unit length of the maize stalk (see U.S. patent application Ser. No. 2004068767 A1, herein incorporated by reference).

In yet another preferred embodiment of the present invention, an isolated polynucleotide comprises (a) a nucleotide sequence encoding a polypeptide associated with stalk mechanical strength, preferably maize stalk mechanical strength, wherein said polypeptide has an amino acid sequence comprising SEQ ID NO:59, or (b) a complement of the nucleotide sequence, wherein the complement and the nucleotide sequence consist of the same number of nucleotides and are 100% complementary.

In another preferred embodiment of the present invention, an isolated polynucleotide comprises SEQ ID NO:61.

A polypeptide is “associated with stalk mechanical strength” in that the absence of the polypeptide in a plant results in a reduction of stalk mechanical strength of the plant when compared to a plant that expresses the polypeptide.

A polypeptide is “associated with maize stalk mechanical strength” in that the absence of the polypeptide in a maize plant results in a reduction of stalk mechanical strength of the maize plant when compared to a maize plant that expresses the polypeptide.

In yet other preferred embodiments of the present invention, a vector comprises a polynucleotide of the present invention, and a recombinant DNA construct comprises a polynucleotide of the present invention, operably linked to at least one regulatory sequence.

Regulatory sequences may include, and are not limited to, promoters, translation leader sequences, introns, and polyadenylation recognition sequences.

Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”. New promoters of various types useful in plant cells are constantly being discovered; numerous examples may be found in the compilation by Okamuro, J. K., and Goldberg, R. B., Biochemistry of Plants 15:1-82 (1989).

A number of promoters can be used in the practice of the present invention. The promoters can be selected based on the desired outcome. The nucleic acids can be combined with constitutive, tissue-specific (preferred), inducible, or other promoters for expression in the host organism. Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

Depending on the desired outcome, it may be beneficial to express the gene from a tissue-specific promoter. Of particular interest for regulating the expression of the nucleotide sequences of the present invention in plants are stalk-specific promoters. Such stalk-specific promoters include the alfalfa stalk-specific S2A gene (Abrahams et al., Plant Mol. Biol. 27:513-528 (1995)) and the like, herein incorporated by reference.

A plethora of promoters is described in WO 00/18963, published on Apr. 6, 2000, the disclosure of which is hereby incorporated by reference. Examples of seed-specific promoters include, and are not limited to, the promoter for soybean Kunitz trysin inhibitor (Kti3, Jofuku and Goldberg, Plant Cell 1:1079-1093 (1989)) β-conglycinin (Chen et al., Dev. Genet. 10:112-122 (1989)), the napin promoter, and the phaseolin promoter.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, Kmiec, U.S. Pat. No. 5,565,350; Zarling et al., PCT/US93/03868), or isolated promoters can be introduced into a plant cell in the proper orientation and distance from a cognate gene of a polynucleotide of the present invention so as to control the expression of the gene. Gene expression can be modulated under conditions suitable for plant growth so as to alter the total concentration and/or alter the composition of the polypeptides of the present invention in plant cell. Thus, the present invention includes compositions, and methods for making, heterologous promoters and/or enhancers operably linked to a native, endogenous (i.e., non-heterologous) form of a polynucleotide of the present invention.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987). Such intron enhancement of gene expression is typically greatest when placed near the 5′ end of the transcription unit. Use of maize introns Adh1-S intron 1, 2, and 6, the Bronze-1 intron are known in the art. See generally, The Maize Handbook, Chapter 116, Freeling and Walbot, Eds., Springer, N.Y. (1994). A vector comprising the sequences from a polynucleotide of the present invention will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers et al., Meth. in Enzymol. 153:253-277 (1987).

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

Preferred recombinant DNA constructs include the following combinations: a) nucleic acid fragment corresponding to a promoter operably linked to at least one nucleic acid fragment encoding a selectable marker, followed by a nucleic acid fragment corresponding to a terminator, b) a nucleic acid fragment corresponding to a promoter operably linked to a nucleic acid fragment capable of producing a stem-loop structure, and followed by a nucleic acid fragment corresponding to a terminator, and c) any combination of a) and b) above. Preferably, in the stem-loop structure at least one nucleic acid fragment that is capable of suppressing expression of a native gene comprises the “loop” and is surrounded by nucleic acid fragments capable of producing a stem.

In another preferred embodiment of the present invention, a recombinant DNA construct of the present invention further comprises an enhancer.

Other preferred embodiments of the present invention include a cell, plant, or seed comprising a recombinant DNA construct of the present invention.

Further, the present invention includes a plant transformed with a recombinant DNA construct of the present invention and having an increased level of stalk mechanical strength when compared to a corresponding nontransformed plant.

Moreover, the following are preferred methods included within the present invention:

A method for transforming a cell, comprising transforming a cell with a polynucleotide of the present invention;

A method for producing a plant comprising transforming a plant cell with a polynucleotide of the present invention, and regenerating a plant from the transformed plant cell;

A method of altering stalk mechanical strength in a plant, comprising (a) transforming a plant, preferably a maize plant, with a recombinant DNA construct of the present invention; and (b) growing the transformed plant under conditions suitable for the expression of the recombinant DNA construct, said grown transformed plant having an altered level (preferably an increased level) of stalk mechanical strength when compared to a corresponding nontransformed plant.

Preferred methods for transforming dicots and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al. (1996) Plant Cell Rep. 15:653-657, McKently et al. (1995) Plant Cell Rep. 14:699-703); papaya (Ling, K. et al. (1991) Bio/technology 9:752-758); and pea (Grant et al. (1995) Plant Cell Rep. 15:254-258). For a review of other commonly used methods of plant transformation see Newell, C. A. (2000) Mol. Biotechnol. 16:53-65. One of these methods of transformation uses Agrobacterium rhizogenes (Tepfler, M. and Casse-Delbart, F. (1987) Microbiol. Sci. 4:24-28). Transformation of soybeans using direct delivery of DNA has been published using PEG fusion (PCT publication WO 92/17598), electroporation (Chowrira, G. M. et al. (1995) Mol. Biotechnol. 3:17-23; Christou, P. et al. (1987) Proc. Natl. Acad. Sci. U.S.A. 84:3962-3966), microinjection, or particle bombardment (McCabe, D. E. et. Al. (1988) BiolTechnology 6:923; Christou et al. (1988) Plant Physiol. 87:671-674).

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In.: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. The regenerated plants may be self-pollinated. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide(s) is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al. (1989) Molecular Cloning: A Laboratory Manual, Cold Spring Harbor Press; Maliga et al. (1995) Methods in Plant Molecular Biology, Cold Spring Harbor Press; Birren et al. (1998) Genome Analysis: Detecting Genes, 1, Cold Spring Harbor, N.Y.; Birren et al. (1998) Genome Analysis: Analyzing DNA, 2, Cold Spring Harbor, N.Y.; Plant Molecular Biology: A Laboratory Manual, eds. Clark, Springer, New York (1997)).

Assays to detect proteins may be performed by SDS-polyacrylamide gel electrophoresis or immunological assays. Assays to detect levels of substrates or products of enzymes may be performed using gas chromatography or liquid chromatography for separation and UV or visible spectrometry or mass spectrometry for detection, or the like. Determining the levels of mRNA of the enzyme of interest may be accomplished using northern-blotting or RT-PCR techniques. Once plants have been regenerated, and progeny plants homozygous for the transgene have been obtained, plants will have a stable phenotype that will be observed in similar seeds in later generations.

Another preferred embodiment included in the present invention is a method for determining whether a plant exhibits a brittle stalk 2 mutant genotype comprising: (a) isolating genomic DNA from a subject; (b) performing a PCR on the isolated genomic DNA using primer pair AGGGAGCTTGTGCTGCTA (SEQ ID NO:53) and GCAGCTTCACCGTCTTGTT (SEQ ID NO:54); and (c) analyzing results of the PCR for the presence of a larger DNA fragment as an indication that the subject exhibits the brittle stalk 2 mutant genotype.

Other preferred embodiments of the present invention include a transgenic plant, preferably maize, whose genome comprises a homozygous disruption of a BRITTLE STALK 2 gene, wherein said disruption comprises an insertion in said gene and results in said transgenic plant exhibiting reduced stalk mechanical strength when compared to its wild type counterpart. Preferably, the disruption comprises the insertion of SEQ ID NO:60.

In another aspect, this invention includes a polynucleotide of this invention or a functionally equivalent subfragment thereof useful in antisense inhibition or cosuppression of expression of nucleic acid sequences encoding proteins associated with stalk mechanical strength, most preferably in antisense inhibition or cosuppression of an endogenous BRITTLE STALK 2 gene.

Protocols for antisense inhibition or co-suppression are well known to those skilled in the art.

Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al. (1998) Plant J. 16:651-659; and Gura (2000) Nature 404:804-808). Another variation describes the use of plant viral sequences to direct the suppression of proximal mRNA encoding sequences (PCT Publication WO 98/36083 published on Aug. 20, 1998). Recent work has described the use of “hairpin” structures that incorporate all, or part, of an mRNA encoding sequence in a complementary orientation that results in a potential “stem-loop” structure for the expressed RNA (PCT Publication WO 99/53050 published on Oct. 21, 1999). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression see Wesley, S. V. et al. (2003) Methods in Molecular Biology, Plant Functional Genomics: Methods and Protocols 236:273-286. A construct where the stem is formed by at least 30 nucleotides from a gene to be suppressed and the loop is formed by a random nucleotide sequence has also effectively been used for suppression (WO 99/61632 published on Dec. 2, 1999). The use of poly-T and poly-A sequences to generate the stem in the stem-loop structure has also been described (WO 02/00894 published Jan. 3, 2002). Yet another variation includes using synthetic repeats to promote formation of a stem in the stem-loop structure. Transgenic organisms prepared with such recombinant DNA fragments have been shown to have reduced levels of the protein encoded by the nucleotide fragment forming the loop as described in PCT Publication WO 02/00904, published 03 January 2002.

The sequences of the polynucleotide fragments used for suppression do not have to be 100% identical to the sequences of the polynucleotide fragment found in the gene to be suppressed. For example, suppression of all the subunits of the soybean seed storage protein β-conglycinin has been accomplished using a polynucleotide derived from a portion of the gene encoding the α subunit (U.S. Pat. No. 6,362,399). β-conglycinin is a heterogeneous glycoprotein composed of varying combinations of three highly negatively charged subunits identified as α, α′ and β. The polynucleotide sequences encoding the α and α′ subunits are 85% identical to each other while the polynucleotide sequences encoding the β subunit are 75 to 80% identical to the α and α′ subunits, respectively. Thus, polynucleotides that are at least 75% identical to a region of the polynucleotide that is target for suppression have been shown to be effective in suppressing the desired target. The polynucleotide may be at least 80% identical, at least 90% identical, at least 95% identical, or about 100% identical to the desired target sequence.

As described above, the present invention includes, among other things, compositions and methods for modulating (i.e., increasing or decreasing) the level of polypeptides of the present invention in plants. In particular, the polypeptides of the present invention can be expressed at developmental stages, in tissues, and/or in quantities which are uncharacteristic of non-recombinantly engineered plants. In addition to altering (increasing or decreasing) stalk mechanical strength, it is believed that increasing or decreasing the level of polypeptides of the present invention in plants also increases or decreases the cellulose content and/or thickness of the cell walls. Thus, the present invention also provides utility in such exemplary applications as improvement of stalk quality for improved stand or silage. Further, the present invention may be used to increase concentration of cellulose in the pericarp (which hardens the kernel) to improve its handling ability. The present invention also may be used to decrease concentration of cellulose in the pericarp (which softens the kernel) to improve its ability to be digested easily.

The isolated nucleic acids and proteins and any embodiments of the present invention can be used over a broad range of plant types, particularly monocots such as the species of the Family Graminiae including Sorghum bicolor and Zea mays. The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Triticum, Bambusa, Dendrocalamus,and Melocanna.

### EXAMPLES

The present invention is further illustrated in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Preparation of cDNA Libraries and Sequencing of Entire cDNA Clones

cDNA libraries representing mRNAs from various maize tissues were prepared as described below. The characteristics of the libraries are described below in Table 1.

TABLE 1
cDNA Libraries from Corn

| | | Clone |
|---|---|---|
| Library | Tissue | (SEQ ID NO:) |
| cbn10 | Corn (Zea mays L.) developing | cbn10.pk0006.f4 |
| | kernel (embryo and endosperm; | (SEQ ID NO: 8) |
| | 10 days after pollination) | |
| cdr1f | Corn (Zea mays, B73) | cdr1f.pk006.d4:fis |
| | developing root (full length) | (SEQ ID NO: 3) |
| cdt2c | Corn (Zea mays L.) developing | cdt2c.pk003.k7 |
| | tassel | (SEQ ID NO: 9) |
| | | cdt2c.pk005.i7a |
| | | (SEQ ID NO: 17) |
| cen3n | Corn (Zea mays L.) endosperm | cen3n.pk0203.g1a |
| | stage 3 (20 days after | (SEQ ID NO: 4) |
| | pollination) normalized* | |
| cest1s | Maize, stalk, elongation | cest1s.pk003.o23 |
| | zone within an internode | (SEQ ID NO: 5) |
| cgs1c | Corn (Zea mays, GasPE Flint) | cgs1c.pk001.d14a |
| | sepal tissue at meiosis about | (SEQ ID NO: 10) |
| | 14-16 days after emergence | |
| | (site of proline synthesis | |
| | that supports pollen development | |
| cr1n | Corn (Zea mays L.) root from | cr1n.pk0144.a2a |
| | 7 day seedlings grown in light | (SEQ ID NO: 11) |
| | normalized* | cr1n.pk0144.a2b |
| | | (SEQ ID NO: 12) |
| csc1c | Corn (Zea mays L., B73) 20 | csc1c.pk005.k4 |
| | day seedling (germination | (SEQ ID NO: 13) |
| | cold stress). The seedling | csc1c.pk005.k4:fis |
| | appeared purple | (SEQ ID NO: 58) |
| ctst1s | Maize, stalk, transition | ctst1s.pk008.I15 |
| | zone. Identify genes that | (SEQ ID NO: 14) |
| | are expressed in the transition | ctst1s.pk014.g20 |
| | zone within an internode | (SEQ ID NO: 15) |
| p0018 | Maize seedling after 10 day | p0018.chsug94r |
| | drought (T001), heat shocked | (SEQ ID NO: 6) |
| | for 24 hrs (T002), recovery | |
| | at normal growth condition | |
| | for 8 hrs, 16 hrs, 24 hrs | |
| p0019 | Maize green leaves (V5-7) | p0019.clwah76ra |
| | after mechanical wounding (1 hr) | (SEQ ID NO: 18) |
| p0032 | Maize regenerating callus, | p0032.crcau13r |
| | 10 and 14 days after auxin | (SEQ ID NO: 7) |
| | removal. Hi-II callus 223a, | |
| | 1129e 10 days. | |
| | Hi-II callus 223a, 1129e 14 days | |
| p008 | Honey N Pearl (sweet corn hybrid) | p0058.chpbr83r |
| | shoot culture. It was initiated | (SEQ ID NO: 16) |
| | on Feb. 28, 1996 from seed | |
| | derived meristems. The culture | |
| | was maintained on 273N medium. | |
| p0102 | Early meiosis tassels, screened | p0102.ceraf50r |
| | 1 (original library P0036) | (SEQ ID NO: 30) |
| | 16-18 cm long. Material was | |
| | cytologically staged and | |
| | determined to contain meiocytes | |
| | in the pachytene stage. | |

*These libraries were normalized essentially as described in U. S. Pat. No. 5,482,845, incorporated herein by reference.

cDNA libraries may be prepared by any one of many methods available. cDNA libraries representing mRNAs from various corn tissues were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (expressed sequence tags or “ESTs”; see Adams, M. D. et al., Science 252:1651 (1991)). The resulting ESTs were analyzed using a Perkin Elmer Model 377 or 3700 fluorescent sequencer.

Full-insert sequence (FIS) data was generated utilizing a modified transposition protocol. Clones identified for FIS were recovered from archived glycerol stocks as single colonies, and plasmid DNAs were isolated via alkaline lysis. Isolated DNA templates were reacted with vector primed M13 forward and reverse oligonucleotides in a PCR-based sequencing reaction and loaded onto automated sequencers. Confirmation of clone identification was performed by sequence alignment to the original EST sequence from which the FIS request was made.

Confirmed templates were transposed via the Primer Island transposition kit (PE Applied Biosystems, Foster City, Calif.) which is based upon the Saccharomyces cerevisiae Ty1 transposable element (Devine and Boeke, Nucleic Acids Res. 22:3765-3772 (1994)). The in vitro transposition system places unique binding sites randomly throughout a population of large DNA molecules. The transposed DNA was then used to transform DH10B electro-competent cells (Gibco BRL/Life Technologies, Rockville, Md.) via electroporation. The transposable element contains an additional selectable marker (named DHFR; Fling and Richards, Nucleic Acids Res. 11:5147-5158 (1983)), allowing for dual selection on agar plates of only those subclones containing the integrated transposon. Multiple subclones were randomly selected from each transposition reaction, plasmid DNAs were prepared via alkaline lysis, and templates were sequenced (ABI Prism dye-terminator ReadyReaction mix) outward from the transposition event site, utilizing unique primers specific to the binding sites within the transposon.

Sequence data was collected (ABI Prism Collections) and assembled using Phred/Phrap. Phred/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies were viewed by the Consed sequence editor (D. Gordon, University of Washington, Seattle; Gordon et al., Genome Res. 8:195-202 (1998)).

Full insert sequence can also be generated by primer walking. Primers can be made from the 5′ or 3′ end of the original EST sequence and reacted with isolated DNA templates from the clone in a PCR-based sequencing reaction and loaded onto automated sequencers. Sequence data can then be collected and further primers made from the ends of these sequences until the full insert sequence is generated. Sequence data can also be assembled and viewed using Sequencher, a software by Gene Codes Corporation (640 Avis Drive, Suite 300, Ann Arbor, Mich. 48108).

### Example 2

### Identification of cDNA Clones

Search for maize cDNA sequences homologous at the nucleic acid and amino acid level to the rice BRITTLE CULM1 (BC1) sequence (SEQ ID NO:1 is the complete coding sequence of the BRITTLE CULM1 gene from rice (NCBI General Identifier No. 34014145); SEQ ID NO:2 is the amino acid sequence of BRITTLE CULM1 from rice (NCBI General Identifier No. 34014146)) was conducted using BLASTN or TBLASTN algorithm provided by the National Center for Biotechnology Information (NCBI) against DuPont's internal proprietary database (Basic Local Alignment Search Tool; Altschul et al., J. Mol. Biol. 215:403-410 (1993); Altschul et al., Nucleic Acids Res. 25:3389-3402 (1997)). DuPont's internal database showed several ESTs homologous at the nucleic acid and protein level, with varying levels of homology (see Table 2). For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

TABLE 2
BLAST Results for Maize Sequences
Homologous to Rice bc1 Gene

| | Blast pLog Score | Blast pLog Score |
|---|---|---|
| Clone | BLASTN | TBLASTN |
| cdr1f.pk006.d4:fis | 9 | 173 |
| SEQ ID NO: 3 | | |
| cen3n.pk0203.g1a | 8 | 93 |
| SEQ ID NO: 4 | | |
| cest1s.pk003.o23 | 8 | 94 |
| SEQ ID NO: 5 | | |
| p0018.chsug94r | 8 | 37 |
| SEQ ID NO: 6 | | |
| p0032.crcau13r | 10 | 93 |
| SEQ ID NO: 7 | | |
| cbn10.pk0006.f4 | 43 | not applicable |
| SEQ ID NO: 8 | | |
| cdt2c.pk003.k7 | 12 | not applicable |
| SEQ ID NO: 9 | | |
| cgs1c.pk001.d14a | 74 | 78 |
| SEQ ID NO: 10 | | |
| cr1n.pk0144.a2a | 127 | 68 |
| SEQ ID NO: 11 | | |
| cr1n.pk0144.a2b | 51 | 32 |
| SEQ ID NO: 12 | | |
| csc1c.pk005.k4 | 62 | not applicable |
| SEQ ID NO: 13 | | |
| ctst1s.pk008.I15 | 152 | 97 |
| SEQ ID NO: 14 | | |
| ctst1s.pk014.g20 | 129 | 68 |
| SEQ ID NO: 15 | | |
| p0058.chpbr83r | 69 | 38 |
| SEQ ID NO: 16 | | |
| cdt2c.pk005.i7a | 84 | 72 |
| SEQ ID NO: 17 | | |
| p0019.clwah76ra | 87 | 75 |
| SEQ ID NO: 18 | | |

Where common or overlapping sequences exist between two or more nucleic acid fragments, the sequences can be assembled into a single contiguous nucleotide sequence, thus extending the original fragment in either the 5-prime or 3-prime direction. Once the most 5-prime EST is identified, its complete sequence can be determined by Full Insert Sequencing (FIS) as described in Example 1.

An FIS was completed on csc1c.pk005.k4 (SEQ ID NO:13). The nucleotide sequence corresponding to the entire cDNA insert in clone csc1c.pk005.k4:fis is shown in SEQ ID NO:58; the amino acid sequence corresponding to the translation of nucleotides 108 through 1451 is shown in SEQ ID NO:59 (nucleotides 1452-1454 encode a stop). The following examples will illustrate that the nucleotide sequence of csc1c.pk005.k4:fis (SEQ ID NO:58) encodes a polypeptide (SEQ ID NO:59) having BRITTLE STALK 2 activity.

### Example 3

### Identification of Maize Genomic Sequences Related to Rice bc1 Gene

Search for maize genomic sequences homologous at the amino acid level to the BRITTLE CULM1 (BC1) sequence (SEQ ID NO:2; NCBI General Identifier No. 34014146) was also conducted using TBLASTN algorithm provided by the National Center for Biotechnology Information (NCBI) against the TIGR Maize genomic assemblies (The TIGR Gene Index Databases, The Institute for Genomic Research, Rockville, Md. 20850; Quackenbush et al., J. Nucleic Acids Res. 28(1):141-145 (2000)). When the sequences were compared a few high scoring hits were identified (Basic Local Alignment Search Tool; Altschul et al., J. Mol. Biol. 215:403-410 (1993); Altschul et al., Nucleic Acids Res. 25:3389-3402 (1997)). These hits are listed in Table 3 with their corresponding P values.

TABLE 3
BLAST Results for Maize Sequences
Homologous to Rice bc1 Gene

| | Blast pLog Score |
|---|---|
| TIGR Assembly Number | TBLASTN |
| AZM2_14907 SEQ ID NO: 19 | 165 |
| AZM2_36996 SEQ ID NO: 20 | 69 |
| AZM2_14120 SEQ ID NO: 21 | 48 |
| AZM2_33700 SEQ ID NO: 22 | 44 |
| OGACO44TC SEQ ID NO: 23 | 37 |
| AZM2_13022 SEQ ID NO: 24 | 26 |
| OGAMW81TM SEQ ID NO: 25 | 24 |
| AZM2_37864 SEQ ID NO: 26 | 18 |

In order to identify the maize homolog/ortholog of the rice bc1 gene, the information that resides in the rice BAC clone was used. The rice BAC clone that was sequenced by Li et al. (OSJNBa0036N23; The Plant Cell 15(9):2020-2031 (2003)) corresponds to BAC clone AC120538 which is part of rice contig 71 on rice chromosome 3. A search of AC120538 sequences to the maize overgo markers (Coe et al., Plant Physiol. 34:1317-1326 (2004)) revealed two hits, both of which are on maize chromosome 7/contig 1599 of DuPont's proprietary maize physical map. One of the sequences on AC120538 has high homology (close to 100%, except for a deletion) to the BC1 protein sequence, and matches maize sequence PCO250027 (74% identity, 86% positives over 98 amino acids) and corresponds to EST p0102.ceraf50r r (SEQ ID NO:30). This EST was not among the high direct hits to bc1 reported in Example 1.

### Example 4

### Characterization of cDNA Clones Encoding BC1-Like Proteins

The maize brittle stalk 2 (bk2) phenotype was first reported in 1940 (Langham, MNL 14:21-22 (1940)), and was mapped by phenotype to chr9L between the markers umc95 and bnl7.13 around the 100 centiMorgan region (Howell et al., MNL 65:52-53 (1991)). To determine which homolog was the most likely candidate for the bk2 locus, the ESTs (including FIS assemblies) and the two highest scoring Genome Survey Sequences (GSS) were aligned and assembled into contigs. A total of three contigs were constructed and these contigs and singeltons are shown in Table 4. PCR primers (see Table 4) were designed from each contig and were then used to amplify from a set of genomic DNA prepared from the oat-maize addition lines (Okagaki, Plant Physiol. 125:1228 (2001)). Each oat-maize addition line contains a full set of the oat chromosomes plus one of the maize chromosome, therefore allowing one to determine the chromosomal positions of the gene simply by PCR reaction. Primers from Contig 1 (SEQ ID NO:27) and AZM2_{—}36996 (SEQ ID NO:20) amplified on maize chromosome 1, while Contig 3 (SEQ ID NO:29) and p0102.ceraf50r (SEQ ID NO:30) mapped to chromosome 7. Contig 2 (SEQ ID NO:28) containing the TIGR GSS sequence AZM2_{—}14907 (SEQ ID NO:19), which was thought to be on chromosome 10 from hybridization data with overgo probes, mapped cleanly to chromosome 9 instead. Since the bk2 locus is on chromosome 9, it was decided to see if this sequence maps to the bk2 region. Contig 1, contig 3, and the EST p0102.ceraf50r (SEQ ID NO:30) (mapped to chromosome 7), were therefore no longer candidates for the bk2 locus.

TABLE 4
Chromosomal Locations of Contigs and Singletons

| | |
|---|---|
| | PCR Primer Pairs |
| Contig or | (5-prime to 3-prime) |

| | | | |
|---|---|---|---|
| Singleton | Left Primer | Right Primer | CL* |

| | | | |
|---|---|---|---|
| Contig 1 - | CACTCCATACAACA | CATTTACCAGGACC | 1 |
| SEQ ID NO:27: | TGCAA | ATCAA | |
| cdr1f.pk006.d4:fis | SEQ ID NO:31 | SEQ ID NO:32 | |
| cen3n.pk0203.g1a | | | |
| cest1s.pk003.o23 | | | |
| p0018.chsug94r | | | |
| p0032.crcau13r | | | |
| Contig 2 - | AACCATACGGGAGC | AAATGCCCTGCCTA | 9 |
| SEQ ID NO:28: | ATCAG | CTGAA | |
| AZM2_14907 | SEQ ID NO:33 | SEQ ID NO:34 | |
| cbn10.pk0006.f4 | | | |
| cdt2c.pk003.k7 | | | |
| cgs1c.pk001.d14a | | | |
| cr1n.pk0144.a2a | | | |
| cr1n.pk0144.a2b | | | |
| csc1c.pk005.k4 | | | |
| ctst1s.pk008.115 | | | |
| ctst1s.pk014.g20 | | | |
| p0058.chpbr83r | | | |
| Contig 3 - | CGAACGGGAACATT | AAGTTCTTGGGCAC | 7 |
| SEQ ID NO:29: | ACCA | CTTGA | |
| cdt2c.pk005.i7a | SEQ ID NO:35 | SEQ ID NO:36 | |
| p0019.clwah76ra | | | |
| SEQ ID NO:20 | TTGCGGAAGTTGAA | ATGGAATGTGACCT | |
| AZM2_36996 | GTTTG | GCAC | |
| | SEQ ID NO:37 | SEQ ID NO:38 | |
| SEQ ID NO:30 | TGACACGGCCATGT | AACCCAAACCGAGG | 7 |
| p0102.ceraf50r | TCTAC | TCTCT | |
| | SEQ ID NO:39 | SEQ ID NO:40 | |

*CL = chromosomal location

### Example 5

### Genetic Mapping of BK2 Candidate

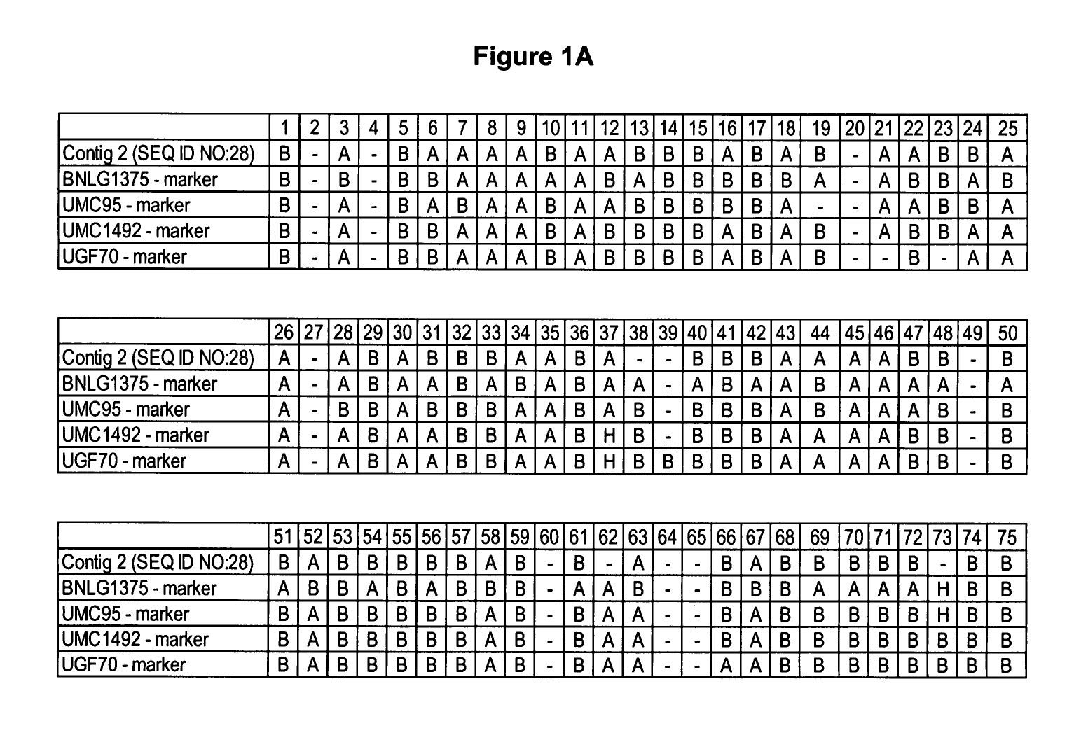

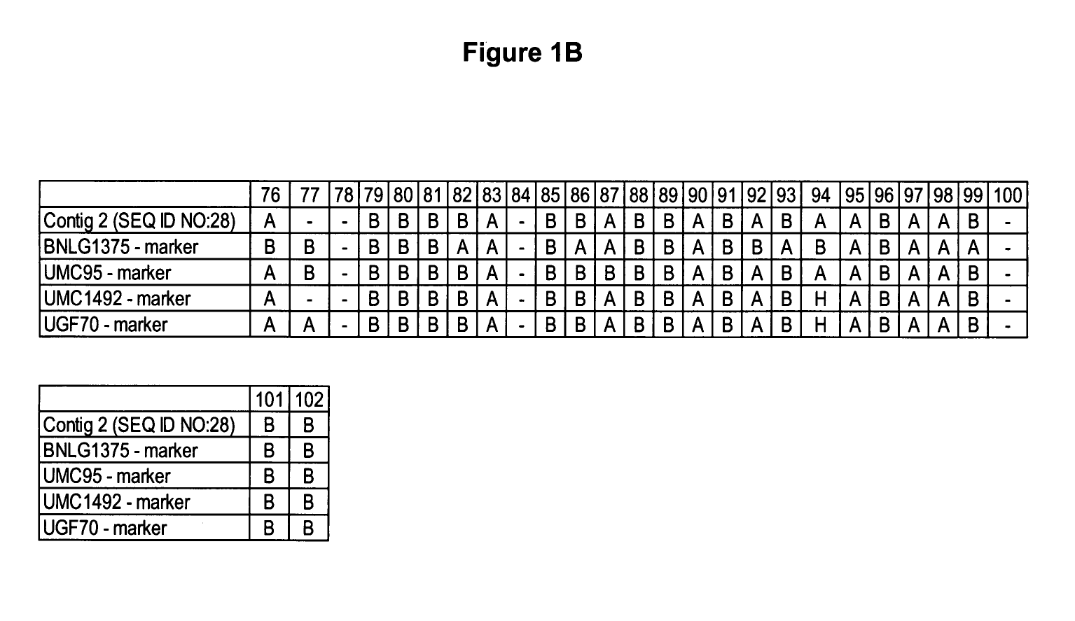

Since bk2 was mapped by phenotype to chr9L between the markers umc95 and bnl7.13 around the 100 centiMorgan region (Howell et al., MNL 65:52-53 (1991)), public PCR-based DNA markers (simple sequence repeats—SSRs) in the vicinity of and including umc95 and bnl7.13 were tested for polymorphism between B73 and Mo17 (parents for intermated B73×Mo17 (IBM) mapping population; see also Maize Genetics and Genomic Database (MaizeGDB)). Single nucleotide polymorphisms (SNPs) were identified between B73 and Mo17 for the locus represented by Contig 2 (SEQ ID NO:28) as described previously by Ching et al. (BMC Genetic 3:19 (2002)). The PCR primers used for Contig 2 were as follows: left primer—AATTAACCCTCACTAAAGGGCATACGGGAGCATCAGTGAG (SEQ ID NO:41); right primer—GTAATACGACTCACTATAGGGCGACGACCTGCMCTCACACTA (SEQ ID NO:42) (5′ to 3′). The left primer has a T3 sequence tagged on the 5′ end to aid in sequencing. Similarly, the right primer has a T7 tag on the 5′ end. DNA amplifications were performed in a 20 μL volume. The reactions contained 20 ng of genomic DNA, 10 pmole (0.2 μM) of each primer, 1× HotStar Taq Master mix from Qiagen and 5% dimethylsulfoxide. The reactions were incubated in a Perkin Elmer 9700 thermocycler with the following cycling conditions: 95° C. for 10 minutes, 10 cycles of 1 minute at 94° C., 1 minute at 55° C., 1 minute at 72° C., 35 cycles of 30 seconds at 95° C., 1 minute at 68° C., followed by a final extension of 7 minutes at 72° C. The PCR products were then converted to a cleaved amplified polymorphic sequence (CAPS) marker by identifying a restriction site polymorphism between the two parents (Konieczny et al., Plant J. 4:403-410 (1993)) Markers showing polymorphism between the two parents were then used to genotype ninety-four individuals from the IBM mapping population. A list of the markers, primers and genotyping methods are listed in Table 5. Genotypic scores (A, B and H where A signifies individuals homozygous for the B73 allele, B is homozygous for the Mo17 allele and H is heterozygous) were then used to map each gene relative to Contig2 (SEQ ID NO:28) obtained from the same segregating population with the software MapMaker (Lander et al., Genomics 1:174-181 (1987)). The genotypic scores can be seen in FIGS. 1A and 1B. The locus represented by Contig 2 (SEQ ID NO:28) was found to lie between umc95 and umc1492, a region where bk2 is believed to be. Thus, the locus sequence for BK2 is most likely represented by the Contig 2 (SEQ ID NO:28).

TABLE 5
Genotyping Method Used for Various Markers

| | | | | Genotyping |
|---|---|---|---|---|
| Marker | Left Primer | Right Primer | Type | Method |
| BNLG1375 | TCGACAACGAGC | CTGCAGATGG | SSR | 4% metaphor |
| | AACTCATC | ACTGGAGTCA | | agarose gel |
| | SEQ ID NO:43 | SEQ ID NO:44 | | |
| UMC95 | AAAGCAACCGAT | TCCGACTTCC | SSR | 1% agarose |
| | TGAtGC | GAGTGAGA | | |
| | SEQ ID NO:45 | SEQ ID NO:46 | | |
| Contig 2 | AATTAACCCTCAC | GTAATACGAC | CAPS | BSAI |
| | TAAAGGGCATACG | TCACTATAGG | | digestion; |
| | GGAGCATCAGTGA | GCGACGACCT | | 1% agarose |
| | SEQ ID NO:41 | GCAACTCACA | | |
| | | CT | | |
| | | SEQ ID NO:42 | | |
| UMC1492 | GAGACCCAACCAA | CTGCTGCAGA | SSR | 4% metaphor |
| | AACTAATAATCTC | CCATTTGAAAT | | |
| | TT | AAC | | |
| | SEQ ID NO:47 | SEQ ID NO:48 | | |
| UFG70 | TGGCTGACGAACT | GATTGCTCAG | SSR | AB1377 |
| | ATTTTCATTCA | TTCATGAGGG | | |
| | SEQ ID NO:49 | AGAT | | |
| | | SEQ ID NO:50 | | |

### Example 6

### Sequencing of the Maize Homolog of Rice bc1 From bk2 Mutant Lines and Wild Type Maize Lines

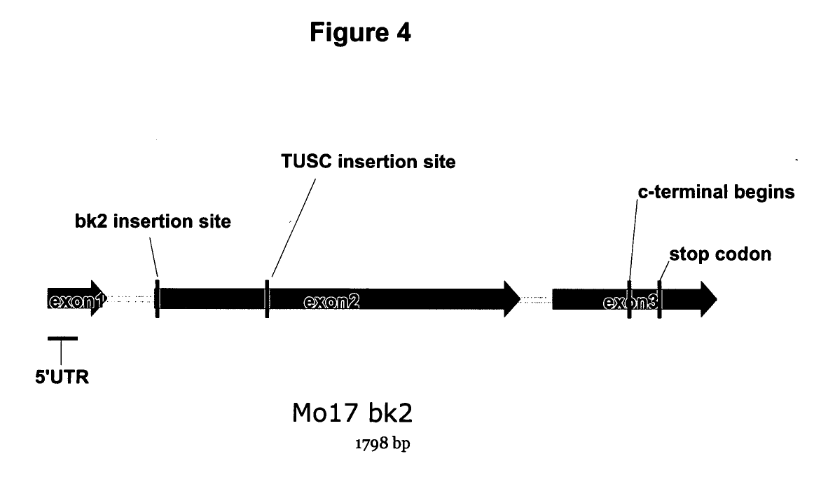

Primers for PCR amplification were designed from Contig 2 (SEQ ID NO:28) (see Table 6 for primers). These primers were used to amplify eight wild type maize germplasms (B73, Mo17, K56, 805, Co159, GT119, Oh43, T218, Tc303, W23). SEQ ID NO:61 is the genomic DNA sequence of the corn BRITTLE STALK 2 gene in Mo17. Putative coding regions are at nucleotide residues 80-158, 286-1269 and 1357-1643 of SEQ ID NO:61 (see FIG. 4). The primers were also used to amplify bk2 brittle mutants (916C, 918K and 918C) obtained from the Maize Genetics COOP Stock Center (USDA/ARS & Crop Sciences/UIUC, S-123 Turner Hall, 1102 S. Goodwin Avenue, Urbana, Ill. 61801-4798). These mutant lines carry the same mutation at the bk2 locus but have a different genetic background (916C has a wx1 background, 918K has a v30 background, and 918C has a wc1 background). Primer set ps238 (SEQ ID NO:53 and SEQ ID NO:54) amplified a product from the bk2 mutants that was approximately 1 kb larger than the amplified product seen in wild type counterparts. The sequences from the mutants were aligned using the Sequencher software (Gene Codes Corporation, Ann Arbor, Mich.) and compared to the eight non-brittle lines to reveal a 1094 base pair insertion (SEQ ID NO:60) in the bk2 mutants at the putative exon2 of the COBRA-like element. The bk2 insertion was found to be between nucleotides 182 and 183 of Contig 2 (SEQ ID NO:28) and between nucleotides 292 and 293 of the MO17 sequence disclosed in SEQ ID NO:61 (indicated as “bk2 insertion site” in FIG. 4). This insertion disrupts the coding region, resulting in a truncated polypeptide and is therefore likely to be the cause of the brittleness in bk2 mutants, further indicating that bk2 is indeed the true homolog of the rice bc1 gene.

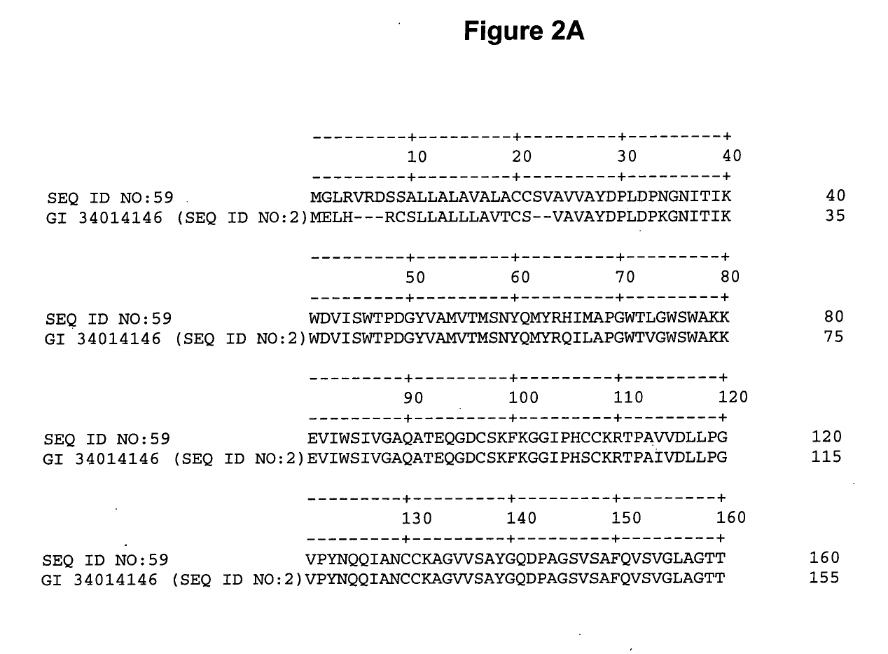

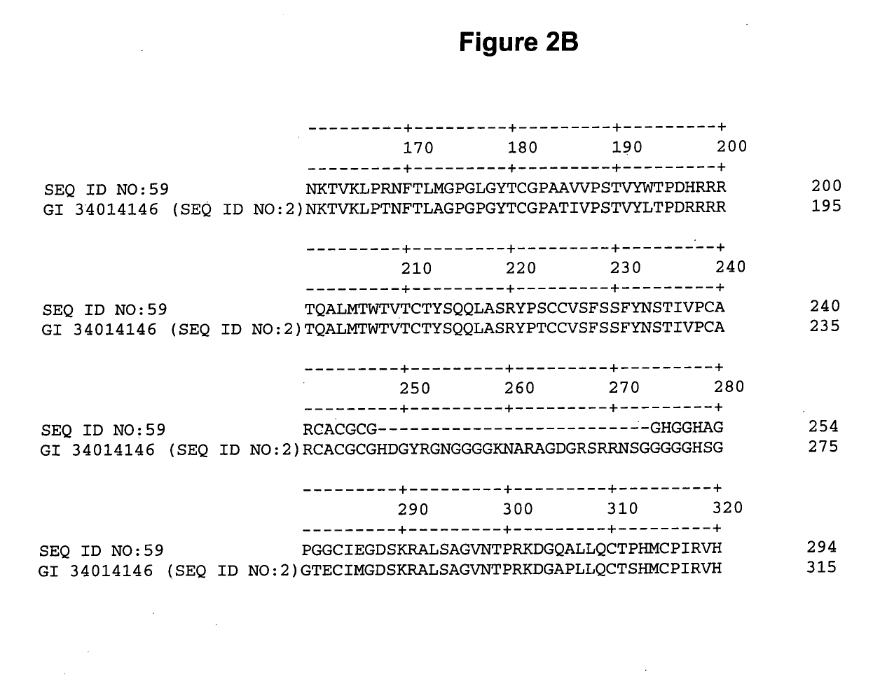

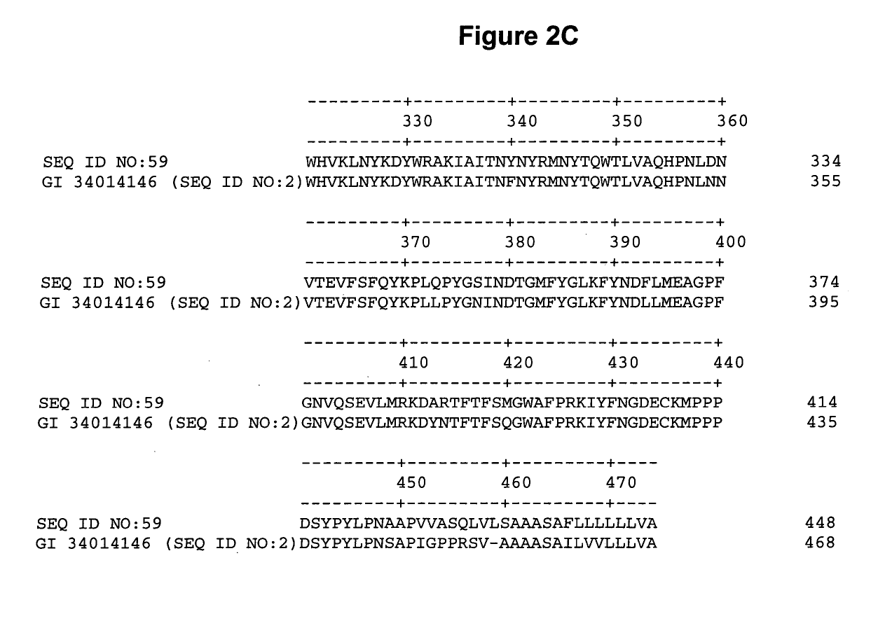

Clone csc1c.pk005.k4:fis (SEQ ID NO:58) encodes a polypeptide (SEQ ID NO:59) having BRITTLE STALK 2 activity. FIGS. 2A-2C show an alignment of the amino acid sequence encoding Zea mays BRITTLE STALK 2 (SEQ ID NO:59) to the amino acid sequence encoding Oryza sativa BRITTLE CULM1 (SEQ ID NO:2). These two amino acid sequences are 84.4% identical using the Clustal V method of alignment with default parameters. The Zea mays BRITTLE STALK 2 cDNA (SEQ ID NO:58) and the Oryza sativa BRITTLE CULM1 cDNA (SEQ ID NO:1) are 66.2% identical using the Clustal V method of alignment with default parameters (data not shown). A PFAM search was conducted on SEQ ID NO:59 using default parameters and yielded a putative phytocheltin synthase-like conserved region at residues 51 to 215 (PFAM score of 340).

TABLE 6

Primer Sequences for
Amplification of bk2 / BK2 Gene

| Primer | | |
|---|---|---|
| Name | Left Primer | Right Primer |
| ps199 | AATTAACCCTCACTAAAGGG | GTAATACGACTCACTATAGGGC |
| | CATACGGGAGCATCAGTGA | GACGACCTGCAACTCACACTA |
| | G | SEQ ID NO:42 |
| | SEQ ID NO:41 | |
| ps231 | AATTAACCCTCACTAAAGGG | GTAATACGACTCACTATAGGGC |
| | CCCTACAACCAGCAGATCG | TGCCAGTGTCATCTGCATT |
| | SEQ ID NO:51 | SEQ ID NO:52 |
| ps238 | AGGGAGCTTGTGCTGCTA | GCAGCTTCACCGTCTTGTT |
| | SEQ ID NO:53 | SEQ ID NO:54 |

*Note: Primers ps199 and ps231 contain a T3 or T7 tag to aid in the sequencing of the resulting PCR products

### Example 7

### Identification of New Alleles of Maize bk2 in TUSC Mutant Population

Full genomic sequence for the putative bk2 locus was used to design primers to screen for Mu-insertion mutants in the TUSC population (U.S. Pat. No. 5,962,764, issued Oct. 5, 1999). The pooled TUSC population was screened with 2 gene primers (CAAGCTMGGAAGGGTCGACATGACG (SEQ ID NO:55) and CGGCTTGTACTGGAAGCTGAAGACCT (SEQ ID NO:56)), each in combination with the Mutator TIR primer (AGAGAAGCCAACGCCAWCGCCTCYATTTCGTC (SEQ ID NO:57)). A single heritable allele, denoted bk2-mu1 was recovered from this screen, and represents an insertion at 302 base pair downstream from the start of the putative exon 2 (between nucleotides 400 and 491 of Contig 2 (SEQ ID NO:28)). The TUSC insertion site in Mo17 is schematically depicted in FIG. 4. Presence of the Mu insertion in the BK2 gene in homozygous F2 progenies from the selected TUSC family co-segregates with the brittle phenotype, as expected. This result can also be confirmed via allelism testing by crossing the bk2 mutant plants in Example 6 to these mutants.

### Example 8

### Prophetic Example Engineering Increased Stalk Strength by Overexpression of Maize BK2 Gene Under a Strong, Stalk-Specific Promoter

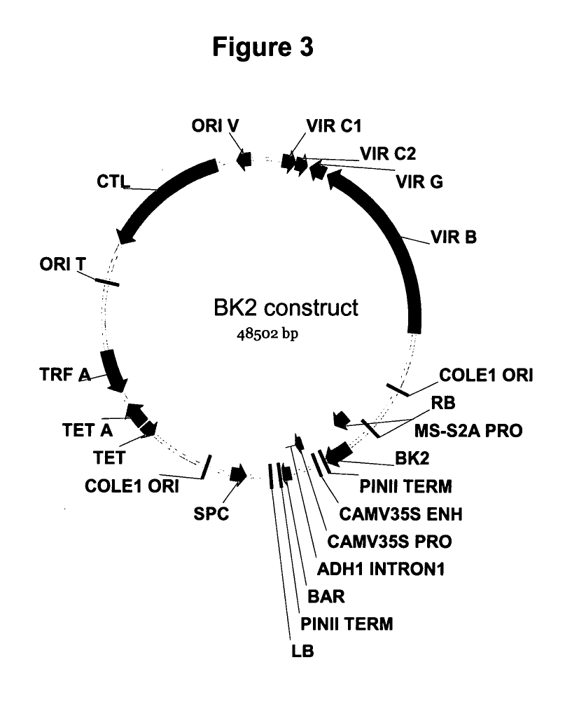

A chimeric transgene is constructed to direct overexpress the BK2 gene/polypeptide in a tissue specific manner. The transgene construct comprises a maize cDNA encoding BK2 (e.g., SEQ ID NO.:58) operably linked to the promoter from the alfalfa stalk-specific S2A gene (Abrahams et al., Plant Mol. Biol. 27:513-528 (1995)). The DNA containing the BK2 ORF is released from the cDNA clone csc1c.pk005.k4:fis by digestion with AccI and StuI. The BK2 ORF is then fused to the S2A promoter on the 5′ end and pinII terminator on the 3′ end to produce an expression cassette as illustrated in FIG. 3. The construct is then linked to a selectable marker cassette containing a bar gene driven by CaMV 35S promoter and a pinII terminator. It is appreciated that one skilled in the art could employ different promoters, 5′ end sequences and/or 3′ end sequences to achieve comparable expression results. Transgenic maize plants are produced by transforming immature maize embryos with this expression cassette using the Agrobacterium-based transformation method by Zhao (U.S. Pat. No. 5,981,840, issued Nov. 9, 1999; the contents of which are hereby incorporated by reference). While the method below is described for the transformation of maize plants with the S2A promoter-BK2 expression cassette, those of ordinary skill in the art recognize that this method can be used to produce transformed maize plants with any nucleotide construct or expression cassette that comprises a promoter linked to maize BK2 gene for expression in a plant.

Immature embryos are isolated from maize and the embryos contacted with a suspension of Agrobacterium, where the bacteria are capable of transferring the S2A promoter-BK2 expression cassette (illustrated above) to at least one cell of at least one of the immature embryos (step 1: the infection step). In this step, the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is included. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus are recovered (step 4: the selection step). Preferably, the immature embryos are cultured on solid medium with a selective agent resulting in the selective growth of transformed cells. The resulting calli are then regenerated into plants by culturing the calli on solid, selective medium (step 5: the regeneration step).

### Example 9

### Prophetic Example Engineering Increased Stalk Strength by Transgenic Expression of Maize BK2 Gene with an Enhancer Element in the Promoter Region

The expression of the BK2 gene is increased by placing a heterologous enhancer element in the promoter region of the native BK2 gene. An expression cassette is constructed comprising an enhancer element such as CaMV 35S fused to the native promoter of BK2 and the full length cDNA. Transgenic maize plants can then be produced by transforming immature maize embryos with this expression cassette as described in Example 8.

### Example 10

### Prophetic Example

### Expression of Recombinant DNA Constructs in Dicot Cells

An expression cassette composed of the promoter from the alfalfa stalk-specific S2A gene (Abrahams et al., Plant Mol. Biol. 27:513-528 (1995)) 5-prime to the cDNA fragment can be constructed and be used for expression of the instant polypeptides in transformed soybean. The pinII terminator can be placed 3-prime to the cDNA fragment. Such construct may be used to overexpress the BK2 gene. It is realized that one skilled in the art could employ different promoters and/or 3-prime end sequences to achieve comparable expression results.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embryos may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic™ PDS1000/HE instrument (helium retrofit) can be used for these transformations.

A selectable marker gene which can be used to facilitate soybean transformation is a chimeric gene composed of the 35S promoter from cauliflower mosaic virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptides and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 11

### Prophetic Example Expression of Recombinant DNA Constructs in Microbial Cells

The cDNAs encoding the instant BRITTLE STALK 2 polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 is constructed by first destroying the EcoRI and HindIII sites in pET-3a at their original positions. An oligonucleotide adaptor containing EcoRI and Hind III sites is inserted at the BamHI site of pET-3a. This creates pET-3aM with additional unique cloning sites for insertion of genes into the expression vector. Then, the NdeI site at the position of translation initiation was converted to an NcoI site using oligonucleotide-directed mutagenesis. The DNA sequence of pET-3aM in this region, 5′-CATATGG, is converted to 5′-CCCATGG in pBT430.

Plasmid DNA containing a cDNA may be appropriately digested to release a nucleic acid -fragment encoding the protein. This fragment may then be purified on a 1% low melting agarose gel. Buffer and agarose contain 10 μg/ml ethidium bromide for visualization of the DNA fragment. The fragment can then be purified from the agarose gel by digestion with GELase™ (Epicentre Technologies, Madison, Wis.) according to the manufacturer's instructions, ethanol precipitated, dried and resuspended in 20 μL of water. Appropriate oligonucleotide adapters may be ligated to the fragment using T4 DNA ligase (New England Biolabs (NEB), Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21 (DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-β-galactoside, the inducer) can be added to a final concentration of 0.4 mM and incubation can be continued for 3 h at 25° C. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

1. An isolated polynucleotide comprising:
(a) a nucleic acid sequence encoding a polypeptide having an amino acid sequence of at least 85% sequence identity, based on the Clustal V method of alignment, when compared to SEQ ID NO:59, wherein expression of said polypeptide in a plant transformed with said isolated polynucleotide results in alteration of the stalk mechanical strength of said transformed plant when compared to a corresponding untransformed plant; or
(b) a complement of the nucleotide sequence, wherein the complement and the nucleotide sequence consist of the same number of nucleotides and are 100% complementary.

2. An isolated polynucleotide comprising:
(a) a nucleic acid sequence encoding a polypeptide having an amino acid sequence of at least 85% sequence identity, based on the Clustal V method of alignment, when compared to SEQ ID NO:59, wherein expression of said polypeptide in a plant exhibiting a brittle stalk 2 mutant phenotype results in an increase of stalk mechanical strength of said plant; or
(b) a complement of the nucleotide sequence, wherein the complement and the nucleotide sequence consist of the same number of nucleotides and are 100% complementary.

3. An isolated polynucleotide comprising:
(a) a nucleotide sequence encoding a polypeptide associated with stalk mechanical strength, wherein said polypeptide has an amino acid sequence comprising SEQ ID NO:59, or
(b) a complement of the nucleotide sequence, wherein the complement and the nucleotide sequence consist of the same number of nucleotides and are 100% complementary.

4. The isolated polynucleotide of claim 1, wherein expression of said polypeptide results in an increase in the stalk mechanical strength.

5. The isolated polynucleotide of claims 1 or 2, wherein said polypeptide has an amino acid sequence of at least 90% sequence identity, based on the Clustal V method of alignment, when compared to SEQ ID NO:59.

6. The isolated polynucleotide of claims 1 or 2, wherein said polypeptide has an amino acid sequence of at least 95% sequence identity, based on the Clustal V method of alignment, when compared to SEQ ID NO:59.

7. The isolated polynucleotide of claims 1 or 2, wherein said polypeptide has an amino acid sequence comprising SEQ ID NO:59.

8. The isolated polynucleotide of claims 1 or 2, wherein said plant is a maize plant.

9. The isolated polynucleotide of claims 1, 2 or 3, wherein said stalk mechanical strength is measured by the three-point bend test.

10. The isolated polynucleotide of claim 3, wherein said polypeptide is associated with maize stalk mechanical strength.

11. A vector comprising the polynucleotide of claims 1, 2 or 3.

12. A recombinant DNA construct comprising the polynucleotide of claims 1, 2 or 3, operably linked to at least one regulatory sequence.

13. A cell comprising the recombinant DNA construct of claim 12.

14. A plant comprising the recombinant DNA construct of claim 12.

15. A seed comprising the recombinant DNA construct of claim 12.

16. The recombinant DNA construct of claim 12, further comprising an enhancer.

17. A method for transforming a cell, comprising transforming a cell with the polynucleotide of claims 1, 2 or 3.

18. A method for producing a plant comprising transforming a plant cell with the polynucleotide of claims 1, 2 or 3, and regenerating a plant from the transformed plant cell.

19. A method of altering stalk mechanical strength in a plant, comprising:
(a) transforming a plant with the recombinant DNA construct of claim 12;
(b) growing the transformed plant under conditions suitable for the expression of the recombinant DNA construct, said grown transformed plant having an altered level of stalk mechanical strength when compared to a corresponding nontransformed plant.

20. The method of claim 18 or 19, wherein said plant is a maize plant.

21. The method of claim 19, wherein said grown transformed plant has an increased level of stalk mechanical strength when compared to a corresponding nontransformed plant.

22. A plant transformed with the recombinant DNA construct of claim 12 and having an increased level of stalk mechanical strength when compared to a corresponding nontransformed plant.

23. A method for determining whether a plant exhibits a brittle stalk 2 mutant genotype comprising:
(a) isolating genomic DNA from a subject;
(b) performing a PCR on the isolated genomic DNA using primer pair AGGGAGCTTGTGCTGCTA (SEQ ID NO:53) and GCAGCTTCACCGTCTTGTT (SEQ ID NO:54); and
(c) analyzing results of the PCR for the presence of a larger DNA fragment as an indication that the subject exhibits the brittle stalk 2 mutant genotype.

24. A transgenic plant whose genome comprises a homozygous disruption of a BRITTLE STALK 2 gene, wherein said disruption comprises an insertion in said gene and results in said transgenic plant exhibiting reduced stalk mechanical strength when compared to its wild type counterpart.

25. A transgenic plant whose genome comprises a homozygous disruption of a BRITTLE STALK 2 gene, wherein said disruption comprises the insertion of SEQ ID NO:60 and results in said transgenic plant exhibiting reduced mechanical strength when compared to its wild type counterpart.

26. An isolated polynucleotide comprising SEQ ID NO:61.


SEQUENCE LISTING (61 sequences)

SEQ ID NO: 1 - DNA, 4498 bp, Oryza sativa
ttttttactaaattaccccttctcctcttccttcaccctcttctatttccactcatcttcctccccatctctctgtgaatctgtttcccgaagcacgggcggtggagaggcctggccacgcgacaaggtgcgtggaggcggaggcctagacaggcccccggcggccggtgcacacggagctggcaagatggtgccacgtgcatatataacaacccatgtggtagtttggtagttgtaggatggtttttaaaaatagtttttttaatcgtccagcacccccccccccccgaggtaccaccgaggtacgaaatctggaccgttcgttcgaattgatctaacggctaggattgcatggtacctcgcggtaccatttttctccttggcgcagtaccgctttggcagtagaggtggaagggtagtttagtctttgaacattagcacgatctgcaccgcatcgcaaaatgccctctgcgccgccgcgttcagctctctgccgcgccgccgcgccaccgtcgggctccgccgcgccgccgcgtcgtgctcagccgcggaatttgacttaaggcgccggcgaggaagtcgcggaggctggggttggagaggcgggtgacctcgaaggtaagcaactcatggtgcttggacgccactggcggcacctccaccttcggcagacggtggaacgtcatggccgggttggccgcggtgacgccggtgaggaaagcccccgttgcaccggtgttgctgtatggtgggtcgacgacgacgacggtgacggcgaggccgcgggcggcgaacaccttgccgaactcgatcatggagaccaggtggcccatgcacgccgctgcttcatgctcagccgcgtcgccgcgtcgggctccaccatgccgccgaatcatgctctgccgcttcgccttgttcacctctctgccgcgccgccacatcgggtttgtctgttccttctctattccgatcctacccccgcttaatcaacggctggattagttttggtactgcgcggtacctgtacctcacggtacaaaacgcaggatggtaacaacactcttttaaaaattaagggagttcttgtttttgtatgttactacagtatatactagtataaaggtaaatgaaaatttctcatcaaaattaagagtggttgcataattttacgaaaaataagaggggttgctgtcaggtgtgatgcttcatcttactgcttggctggatcatcggagaggaatgaatggttccgtgctttctacttctactgaactcgtattgtgtataagtgcatcacgcacgcaagtaagtaaagtacgtacttacacaggaatatgtacgtacgtacgtacggcagatggagaaggatgcatatgcgatcgatgaggttggcgttcgtgttgaaaaaacgtgccaactggttggttgaggaatatcaaaatccttgtccactttgtaagccagggatagtcgtaccgccaaacagaagtatgatggaaaagcaagtaacagaatctaatgacatcaattataatcacgtcaggtatagagcgagcggtagcagatcgagtatccatgacacgatccatcgatctcgcgttggcctggcctgaccgtaatctatggtattttgacatccaatgatcaccaatttgattgttttattattttaaatcttcagtactaatataaagtgattgatgaagaaaacaaatttgatagtcatatatacatgtcgtcggtggctgcagaggcggtgatcgatcaaacgttgcaactggcggaacagatgccgcgcaccttacacgaacgaaaaattggcaaaatgttccgccgtcgctatcgcaaacacaccctcttctctcctggttcgatcgatgaggtgagcgcgcgagatctccggcgtccctttccctccgtcaccatcaaccacggttgcttcgcccagccgcgatgccgcagccgcaggccgtccaaatcatcagcttcacagaccagccagacgagtgtgcagagcgagcgccatgcccgcatatgcacgggacgaacccaagattcacggcatgttaaccatgtcggagaggtggcgctgagccatcaccccttccgtcatgcaatgagtcctcctcaagaaacccaaccgacgatcaatccatcgaggtgtgacgcgccatctcgccgctcggtggcttcttcttcttctaccttctcctccctcttcctggccagccagtgcacgccttctcattcaattccctgctcacctcgatcgagtagctgctgctgctgtgctagcttgctcgccggccggtgaggtcgacgatggagctgcacagatgctctctcctcgctctgctcctcgccgtgacatgctcggttgcaggttaattacttcttcgatcttcttgcccattattcctaattaaattatacttttgctgttgattaatcaatcatgcatgtgtgtgtgcttgcagtggcgtatgatccgctggacccgaaggggaacatcacgataaagtgggacgtgatatcgtggacgcccgacgggtacgtggcgatggtgacgatgagcaactaccagatgtaccggcagatcctggcgcccgggtggacagtggggtggtcgtgggccaagaaggaggtcatctggtccatcgtgggggcccaggccaccgagcagggcgactgctccaagttcaagggcggcatcccccacagctgcaagcgcaccccggccatcgtcgacctcctccccggcgtcccctacaaccagcagatcgccaactgctgcaaggccggcgtcgtctccgcctacggccaggaccccgccggatccgtctccgccttccaggtctccgtcggcctcgccggcaccaccaacaagaccgtcaagctacccaccaacttcaccctcgccggcccgggacccgggtacacgtgtggcccggccaccatcgtcccttccaccgtctacctcaccccggaccggcgccgccgcacccaggcgctcatgacgtggaccgtcacctgcacctactcccagcagctggcgtcgcgctacccgacctgctgcgtctccttctcctccttctacaacagcaccatcgtgccgtgcgccaggtgcgcctgcgggtgcggccacgacggctaccgcggcaacggcggcggcgggaagaacgcccgcgccggcgacggacgcagcagacgcaacagcggcggcggcggagggcacagcggcggcaccgagtgcatcatgggcgactcgaagcgggcgctgtcggcgggggtgaacacgccgcgcaaggacggggcgccgctgctgcagtgcacgtcgcacatgtgcccgatccgcgtgcactggcacgtcaagctcaactacaaggactactggcgcgccaagatcgccatcacaaacttcaactaccgcatgaactacacccagtggacgctcgtcgcccagcaccccaacctcaacaacgtcaccgaggtcttcagcttccagtacaagcccctcctcccctacggcaacatcagtaagctctctaccacaacctcttattcctcctctccgacatcgttctcgctttcatatctatacctgtactaattggacgacaccacggccatggtatattgcagacgacaccggcatgttctacgggctcaagttctacaacgacctgctcatggaggcagggccgttcggcaacgtgcagtcggaggtgctgatgcgaaaggactacaacaccttcaccttcagccagggctgggcgttcccgcgcaagatctacttcaacggcgacgagtgcaagatgccgccgccggactcctacccctacctacccaactccgctccgatcgggccgccgcgttccgtggccgccgccgcctcggcgatcttggtggtgctcctcctggtggcatgatcagaaaaatgtccccttttgctttgtcttcttgataattcccacatgtttggagagcagtgtaggtaggggcattttggtctattcatactggatattcagtcaaagaggaaatctgtgatattgtgttaactttgaaattgcctgatagatctccataatgtacaacacaatcaggctggaagagttttggtcagtccccagttaggccagccctgagaaatcacaccacaaacttttctgcaaattctgttgtgactacaaatatgtatgcaggtattgaccttgaattgagaggaaaaaagaaacaatttccacatttactgaccaactacaaaatgcaatttcttgcaatcagatgagatggcaaacatttctctagacaattaatgttgggacttggggttctcaattagtcttcacacttcagaccaagaatacacaccatcagaatgtacaacccaaactttaatgatttcgaggaacctaaacttacaacctaaatcaaacgcgaattagcttttcatgcaagagcacaccctaaacttccaaaagactcagtatgtcaa

SEQ ID NO: 2 - protein, 468 aa, Oryza sativa
MELHRCSLLALLLAVTCSVAVAYDPLDPKGNITIKWDVISWTPDGYVAMVTMSNYQMYRQILAPGWTVGWSWAKKEVIWSIVGAQATEQGDCSKFKGGIPHSCKRTPAIVDLLPGVPYNQQIANCCKAGVVSAYGQDPAGSVSAFQVSVGLAGTTNKTVKLPTNFTLAGPGPGYTCGPATIVPSTVYLTPDRRRRTQALMTWTVTCTYSQQLASRYPTCCVSFSSFYNSTIVPCARCACGCGHDGYRGNGGGGKNARAGDGRSRRNSGGGGGHSGGTECIMGDSKRALSAGVNTPRKDGAPLLQCTSHMCPIRVHWHVKLNYKDYWRAKIAITNFNYRMNYTQWTLVAQHPNLNNVTEVFSFQYKPLLPYGNINDTGMFYGLKFYNDLLMEAGPFGNVQSEVLMRKDYNTFTFSQGWAFPRKIYFNGDECKMPPPDSYPYLPNSAPIGPPRSVAAAASAILVVLLLVA

SEQ ID NO: 3 - DNA, 2102 bp, Zea mays
ggaaagcagcgctgcggagcagagtgtgtcgcttcgctgtaaaaacaggggagagggagacgcgcccgctgccagtgcctgccgcacacgcgtttagcgtttaagttccactcctcgccgccccagatctccgccctcctcaccactgcccctcattccccggcgcccagcacccggcggccgcaaccgccgcagtccggagcaagatcggcgggtagacggacggacggacgggcgacaggcgggcgggcgcggctctgtctgtatctatctgttggtgggagaccggttgtgtcggttaggcggcggcgggtgggaaggaagaatggcggcgggcggcagatccatcgcgtgctttgccgccgtgctgctcgcggccgcgctgctcctctccgcaccgaccaccacagaggcctacgattcgctggatccaaacggcaacatcactataaaatgggatatcatgcagtggactcctgacggatatgtcgctgttgtcacaatgttcaattatcaacaatttcggcacatcggggcacctggatggcagcttgggtggacatgggcaaaaaaggaggttatatggtcaatggttggggctcagaccactgaacagggtgactgctcaaagttcaagggcaacaccccccattgctgcaagaaagatccaacaattgttgatttacttccaggcactccatacaacatgcaaattgccaattgctgcaaggcaggagttataaatacctttaaccaggacccagcaaatgctgcttcctccttccagatcagtgttggtcttgctggaactaccaataaaactgttaaggtgccgaagaatttcactcttaagactccaggccctgggtacacatgtgggcgtgctattgttggcaggccaacgaagtttttctctgcagatgggcgcagggtaacccaagctctaatgacatggaatgtgacctgcacatattcccaatttcttgctcagaagactccatcctgctgtgtatctctctcatcattttataatgacacaattgtgaactgcccgacatgctcatgtggctgccagaacccaagtgggtcaaactgtgtgaacgaggattcacctaatctacaagccgcaattgatggtcctggtaaatggactggccagcctcttgtacaatgcacttctcacatgtgcccaataagaatccactggcatgtgaagctcaactacaaggaatactggagagtgaaaatcactatcacgaacttcaacttccgcatgaattacacacagtggaacttagttgctcagcatccaaactttgataatatcactcagttgttcagcttcaactacaaaccacttactccatatgggggtggcataaatgatacggcaatgttctggggtgtaaagttctacaatgatttgctgatgcaagccggcaaacttgggaatgtgcaatcagaactgcttctccgcaaggactcacggactttcacattcgaaaagggatgggccttcccacgccgagtgtacttcaatggtgataattgtgtcatgccatctcctgaaaattatccatggctgccgaatgcaagccctctaacaaaacaagcattgacactcccactcttgatattctgggttgccttggctgttctgttggcttatgcatgatgagtgggatcaagatgtttagcaagcttcaagttgatgtcggattccatgaggtgcactgcaacgggatatttattcattcaattccatagcggcacaggagagatgaggcgaagccaagaaaaagtggatgtgtgtgtgtgtgtgtttgtaagttaaagggccaaaatgtatttcttgtctggtagtatatagcagctctacaacactttggtgaacttagttactgcaaattaggcaattacagttgcaccttttgtattttatagcaaacccagacttctattggattctatgactgcccctcttgtagtaaacgcaaggcttcactggtactcctgtttaaagattggtcaaatagaagagacgacggtgattgtcaaaaaaaaaaaaaaaaaaaaaaaaaaaaaaaaaaaaaaaaaaaaaa

SEQ ID NO: 4 - DNA, 680 bp, Zea mays
  misc_feature (673)..(678): n = a, c, g or t
caaatggcaacatcaccataaaatgggatatcatgcagtggactcctgatggatatgtcgctgttgtcacaatgtttaattatcaacaatttcggcatatcggcgcacctggttggcagcttgggtggacatgggcaaagaaggaggttatatggtcaatggttggggctcagaccactgaacagggcgactgctcaaagttcaagagcagcccaccccattgctgcaagaaagatccaacaattgtcgatttacttccaggcactccatacaacatgcaaattgccaattgctgcaaggcaggagttgtaaatacctttaaccaggacccagcaaatgctgcttcctccttccagatcagtgttggtcttgctggaactaccaataaaactgttaaggtgcccaggaacttcactcttaagactccaggccctgggtacacatgtgggcgtgccattgttggcaggcctacgaagtttttcaccgcggacgggcgcagggcaacccaagctctaatgacatggaatgtgacctgcacatattcccaatttcttgctcagaagactccatcctgctgtgtatctctatcatcgttttataatgacacaattgtgaactgcccaacatgctcatgtggctgccagaacccaagtgggtcaaactgtgtgaatgnnnnnncn

SEQ ID NO: 5 - DNA, 678 bp, Zea mays
  misc_feature (600)..(605): n = a, c, g or t
ccacgcgtccgctgcaacagaggcttatgattcgctggatccaaatggcaacatcaccataaaatgggatatcatgcagtggactcctgatggatatgtcgctgttgtcacaatgtttaattatcaacaatttcggcatatcggcgcacctggttggcagcttgggtggacatgggcaaagaaggaggttatatggtcaatggttggggctcagaccactgaacagggcgactgctcaaagttcaagagcagcccaccccattgctgcaagaaagatccaacaattgtcgatttacttccaggcactccatacaacatgcaaattgccaattgctgcaaggcaggagttgtaaatacctttaaccaggacccagcaaatgctgcttcctccttccagatcagtgttggtcttgctggaactaccaataaaactgttaaggtgcccaggaacttcactcttaagactccaggccctgggtacacatgtgggcgtgccattgttggcaggcctacgaagtttttcaccgcggacgggcgcagggcaacccaagctctaatgacatggaatgtgacctgcacatattcccaatttcttgctcnnnnnncncnatcctgctgtgtatctctatcatcgttttataatgacacaattgtgaactgcccaacatgctcatgtnn

SEQ ID NO: 6 - DNA, 462 bp, Zea mays
  misc_feature (337)..(337): n = a, c, g or t
gcaatttcggcatatcggcgcacctggttggcagcttgggtggacatgggcaaagaaggaggttatatggtcaatggttggggctcagaccactgaacagggcgactgctcaaagttcaagagcagcccaccccattgctgcaagaaagatccaacaattgtcgatttacttccaggcactccatacaacatgcaaattgccaattgctgcaaggcaggagttgtaaatacctttaaccaggacccagcaaatgctgcttcctccttccaagatcaagtgtttggtcttgctgggaactaacaattaaaactgttaaggtggcccaggaacttcaantcttaagaatccaaggcctggggtacaacatgttgggcgtgcaattgtttggaaggctacgaagttttcaccgncgancgggcgcaagggnaaccaaagtctaatgacaatggatggactgcaca

SEQ ID NO: 7 - DNA, 372 bp, Zea mays
  misc_feature (128)..(129): n = a, c, g or t
ggccctgggtacacatgtgggcgtgctattgttggcaggccaacaaagtttttcactgcggatgggcgcagggtaacccaagctctaatgacatggaatgtgacctgcacatattcccaatttcttgnncagaagactccgtcctgctgtgtatctctctcatcattttataatgacacaattgtgaactgcccgacatgctcatgtggctgccagaacccaagtgggtcaaactgtgtgaacgaggattcacctaatctacaagccgcaattgatggtcctggtaaatggactggccagcctcttgtacaatgcacttctcagatgtgcccaataagaatccactgggcatgtgaagctcaactacaagga

SEQ ID NO: 8 - DNA, 501 bp, Zea mays
  misc_feature (128)..(128): n = a, c, g or t
acgcaaggaccttcaccttcagcatgggctgggcgttcccgcgcaagatctacttcaacggcgacgagtgcaagatgccgccgccggactcctacccctacctgcccaacgccgcgcccgtcgtcgcntcgcagctggtcctgtccgccgccgcctcggcgttcctactgttgctgctcctggtggcatgaccgtgaccgaaccaagggcaaggcctccgttttgttttcccgtctcgtcccgtgggcagggagcagacttcagtaggcagggcattttatttggtttttttgccaaggattcaacacttgggttttcgtcagaggaaaactgtcgtgtatgtagtgtgagttgcaggtcgtcggatccccacgtacaagacaatctttggatctagaatatgcaaaacgtgaatcagcacgccaggatcatcgtctcctacaagattggcaaaaaaaaaaaaaaaaaaaaaaaaaaaaaaaactcgagactagttctctc

SEQ ID NO: 9 - DNA, 364 bp, Zea mays
  misc_feature (1)..(1): n = a, c, g or t
ncgtncgggcaggatccggcggggtccgtctccgcgttccaaggtctccgtcggcctggccggtaccaccaacaagacggtgaagctgcccaggaacttcacgctcatggggcccgggctgggctacacctgcnggcccgccgccgtggcgccgtccaccgtgtactggacgcccgacnaccggcgccggacgcaggcgcctcatgacgtggacggtgacctgcacctactnctcaagcaagctggngtcccggtacccgtcttgctgcgtctccttctcctccttctacaaacaancaccaattcgttgccgtgccgcccggtgacgcgttgcgggctgnccggtntgnccangggagggtaa

SEQ ID NO: 10 - DNA, 640 bp, Zea mays
  misc_feature (607)..(609): n = a, c, g or t
ccacgcgtccgctggcacgtcaagctcaactacaaggactactggcgcgccaagatcgccatcaccaactacaactacaggatgaactacacgcagtggacgctggtggcgcagcaccccaacctggacaacgtcaccgaggtcttcagcttccagtacaagccgctgcaaccatacgggagcatcaatgacactggcatgttctacgggctcaagttctacaacgactttctcatggaggccggcccgttcggcaacgtgcagtcggaggtgctcatgcgcaaggacgcaaggaccttcaccttcagcatgggctgggcgttcccgcgcaagatctacttcaacggcgacgagtgcaagatgccgccgccggactcctacccctacctgcccaacgccgcgcccgtcgtcgcctcgcagctggtcctgtccgccgccgcctcggcgttcctactgttgctgctcctggtggcatgaccgtgaccgaaccaagggcaaggcctccgttttgttttcccgtctcgtcccgtgggcagggagcagacttcagtaggcagggcattttatttggttttgccaaggattcaacacttgggttttcgtcagnnnaaaactgtcgtgtatgtagtgtgagttgcan

SEQ ID NO: 11 - DNA, 693 bp, Zea mays
  misc_feature (21)..(23): n = a, c, g or t
cctggacaacgtcaccgaggnnntcagcttccagtacaagccgctgcaaccatacgggagcatcaatgacactggcatgtnctacgggctcaagttctacaacgactttctcatggaggccggcccgttcggcaacgtgcagtcggaggtgctcatgcgcaaggacgcaaggaccttcaccttcagcatgggctgggcgttcccgcgcaagatctacttcaacggcgacgagtgcaagatgccgccgccggactcctacccctacctgcccaacgccgcgcccgtcgtcgcctcgcagctggtcctgtccgccgccgcctcggcgttcctactgttgctgctcctggtggcatgaccgtgaccgaaccaagggcaaggcctccgttttgttttcccgtctcgtcccgtgggcagggagcagacttcagtaggcagggcattttatttggtttttttgccaaggattcaacacttgggttttcgtcagaggaaaactgtcgtgtatgtagtgtgagttgcaggtcgtcggatccccacgtacaagacaatctttggatctagaatatgcaaaacgtgaatcagcacgccaggatcatcgtctcctacaagattggcagaaaaaaaatctcatgatgagtgatgtgtcaacagacctatatatatgtgataatcactggtttcaacggttgcctg

SEQ ID NO: 12 - DNA, 603 bp, Zea mays
  misc_feature (394)..(396): n = a, c, g or t
caggcaaccgttgaaaccagtgattatcacatatatataggtctgttgacacatcactcatcatgaaattttttttctgccaatcttgtaggaaacgatgatcctggcgtgctgattcacgttttgcatattctaaatccaaagattgtcttgtacgtggggatccgacgacctgcaactcacactacatacacgacagttttcctctgacgaaaacccaagtgttgaatccttggcaaaaaaaccaaataaaatgccctgcctactgaagtctgctccctgcccacgggacgagacgggaaaacaaaacggaggccttgcccttggttcggtcacggtcatgccaccaggagcagcaacagtaggaacgccgaggcggcggcggacaggaccnnntgcgaggcgacaacgggcgcggcgttgggcaggtaggggtaggagtccggcggcggcatcttgcactcgtcgccgttgaagtaaatcttgcgcgggaacgcccagcccatgctgaaggtgaaggtccttgcgtccttgcgcatgagnacctccgactgcacgttgccgaacgggccggcctccatgnnnangtcgttgtnnnacttg

SEQ ID NO: 13 - DNA, 474 bp, Zea mays
  misc_feature (307)..(307): n = a, c, g or t
gggatcggagcttgtgctgctactgctactataccagcgctagctagcagcagccgccggccggctcgcgcaagctaaggaagggtcgacatgacgatggggctccgcgtccgcgactcctccgcgctgctggctctggccgtcgcgctcgcctgctgctccgttgcagtggtggcctacgaccccctggacccgaacggcaacatcaccatcaagtgggacgtgatctcgtggacgcccgacgggtacgtggcgatggtgacgatgagcaactaccagatgtaccgggcacatcatggcgcccggntggacgttggggtggtcgtgggccaagaaggagggtgatctggtccatcgtgggggcgcaagccacggaagcaagggggactgctcccangtttcaaggggcgggcatcccgcactgctgcaagcncaaccccggccggtggtgggacctcctncccgggggngncc

SEQ ID NO: 14 - DNA, 686 bp, Zea mays
  misc_feature (560)..(561): n = a, c, g or t
ccacgcgtccggcgctagctagcagcagccgccggccggctcgcgcaagctaaggaagggtcgacatgacgatggggctccgcgtccgcgactcctccgcgctgctggctctggccgtcgcgctcgcctgctgctccgttgcagtggtggcctacgaccccctggacccgaacggcaacatcaccatcaagtgggacgtgatctcgtggacgcccgacgggtacgtggcgatggtgacgatgagcaactaccagatgtaccggcacatcatggcgcccgggtggacgttggggtggtcgtgggccaagaaggaggtgatctggtccatcgtgggggcgcaggccacggagcagggggactgctccaagttcaagggcggcatcccgcactgctgcaagcgcaccccggccgtggtggacctcctcccgggggtgccctacaaccagcagatcgccaactgctgcaaggccggcgtggtgtcggcgtacgggcaggacccggcggggtccgtctccgcgttccaggtctccgtcggcctggccggtaccaccaacaagacnntgaagctnnncaggaacttcacgctcatggggcccgggctgggctacacctgcgggcccgncgccgtggtgccgtccaccgtgtactggacgcccgaccaccggcgccgnanncnnncgctcatg

SEQ ID NO: 15 - DNA, 530 bp, Zea mays
  misc_feature (32)..(33): n = a, c, g or t
ccacgcgtccggctgctactgctactataccnncgctagctagcagcagccgccggccggctcgcgcaagctaaggaagggtcgacatgacgatggggctccgcgtccgcgactcctccgcgctgctggctctggccgtcgcgctcgcctgctgctccgttgcagtggtggcctacgaccccctggacccgaacggcaacatcaccatcaagtgggacgtgatctcgtggacgcccgacgggtacgtggcgatggtgacgatgagcaactaccagatgtaccggcacatcatggcgcccgggtggacgttggggtggtcgtgggccaagaaggaggtgatctggtccatcgtgggggcgcaggccacggagcagggggactgctccaagttcaagggcggcatcccgcactgctgcaagcgcaccccggccgtggtggacctcctcccgggggtgccctacaaccagcagatcgccaactgctgcaaggccggcgtggtgtcggcgtacgggcagnacccggcgnnntcc

SEQ ID NO: 16 - DNA, 260 bp, Zea mays
  misc_feature (113)..(113): n = a, c, g or t
gcgcgcaggccacggagcagggggactgctccaagttcaagggcggcatcccgcactgctgcaagcgcaccccggccgtggtggacctcctcccgggggtgccctacaaccancagatcgccaactgctgcaaggccggcgtggtgtcggcgtacgggcaggacccggcggggtccntctccgcgttccaggtctccgtcggcctctccggcaccaccaacaagacggtgaagctgnccaggaanttnacgctcatnggg

SEQ ID NO: 17 - DNA, 513 bp, Zea mays
  misc_feature (503)..(506): n = a, c, g or t
gcacgagagtgcatgcacgcccgatactgctagccaaggccaagccagtgcaggcgcggtggtgtgtgttgttctcgtcgcgcactcgccggcagcgatggagccccgccgctccgtgctgctcctggccctcgccgtcgccgccgcgctctccgtcgcagtggcttacgacccgttggacccgaacgggaacattaccatcaagtgggacatcatgtcgtggacgcccgacggctatgtcgcggtggtgaccatcaacaacttccagacgtaccggcagatcacggcgccggggtggacggtggggtggacgtgggcgaagcgggaggtgatctggtccatggtgggcgcgcaggccacggagcagggcgactgctcccgcttcaaggccaacatcccgcactgctgcaagcgcaccccggccgtcgtcgacctgctccccggcgtgccctacaaccagcagatcgccaactgctgccgcggcggcgtcgtcagcgcctacnnnnangacnc

SEQ ID NO: 18 - DNA, 599 bp, Zea mays
  misc_feature (478)..(479): n = a, c, g or t
tgcacgcccgatactgctagccaaggccaagccagtgcaggcgcggtggtgtgtgttgttctcgtcgcgcactcgccggcagcgatggagccccgccgctccgtgctgctcctggccctcgccgtcgccgccgcgctctccgtcgcagtggcttacgacccgttggacccgaacgggaacattaccatcaagtgggacatcatgtcgtggacgcccgacggctatgtcgcggtggtgaccatcaacaacttccagacgtaccggcagatcacggcgccggggtggacggtggggtggacgtgggcgaagcgggaggtgatctggtccatggtgggcgcgcaggccacggagcagggcgactgctcccgcttcaaggccaacatcccgcactgctgcaagcgcaccccggccgtcgtcgacctgctccccggcgtgccctacaaccagcagatcgccaactgctgccgcggcggcgtcnncagcgcctacggccaggacccggccaccgccgtcgccgcgttccaggtcagcgtcggccaggccggcaccaccaaccgcaccgtcaaggtgcccaagaacttccnnngctnnggnnnnng

SEQ ID NO: 19 - DNA, 1530 bp, Zea mays
cacggagcagggggactgctccaagttcaagggcggcatcccgcactgctgcaagcgcaccccggccgtggtggacytcctcccgggggtgccctacaaccagcagatcgccaactgctgcaaggccggcgtggtgtcggcgtacgggcaggacccggcggggtccgtctccgcgttccaggtctccgtcggcctggccggtaccaccaacaagacggtgaagctgcccaggaacttcacgctcatggggcccgggctgggctacacctgcgggcccgccgccgtggtgccgtccaccgtgtactggacgcccgaccaccggcgccggacgcaggcgctcatgacgtggacggtgacctgcacctactcgcagcagctggcgtcccggtacccgtcctgctgcgtctccttctcctccttctacaacagcaccatcgtgccgtgcgcccggtgcgcgtgcggctgcggcggccacggcggccacgcgggtccgggcggctgcatcgagggggactccaagcgcgcgctgtcggccggggtgaacacgccgcgcaaggacggccaggcgctgctgcagtgcacgccgcacatgtgccccatccgggtgcactggcacgtcaagctcaactacaaggactactggcgcgccaagatcgccatcaccaactacaactacaggatgaactacacgcagtggacgctggtggcgcagcaccccaacctggacaacgtcaccgaggtcttcagcttccagtacaagccgctgcaaccatacgggagcatcagtgagtataatcatcgtcatctgatgacatgacatgacatgtacataatcatcggtgtctcaaatatatatatgcaattaatgcagatgacactggcatgttctacgggctcaagttctacaacgactttctcatggaggccggcccgttcggcaacgtgcagtcggaggtgctcatgcgcaaggacgcaaggaccttcaccttcagcatgggctgggcgttcccgcgcaagatctacttcaacggcgacgagtgcaagatgccgccgccggactcctacccctacctgcccaacgccgcgcccgtcgtcgcctcgcagctggtcctgtccgccgccgcctcggcgttcctactgttgctgctcctggtggcatgaccgtgaccgaaccaagggcaaggcctccgttttgttttcccgtctcgtcccgtgggcagggagcagacttcagtaggcagggcattttatttggtttttttgccaaggattcaacacttgggttttcgtcagaggaaaactgtcgtgtatgtagtgtgagttgcaggtcgtcggatccccacgtacaagacaatctttggatctagaatatgcaaaacgtgaatcagcacgccaggatcatcgtctcctacaagattggcagaaaaaaaatctcatgatgagtgatgtgtcaacagacctatatatatgtgataatcactggtttcaacggttgcctgaacatttgctaacccatcagtagccactact

SEQ ID NO: 20 - DNA, 1101 bp, Zea mays
gtttggatgcttggctactaagttccactgcgtgtaattcttgcggaagttgaagtttgtgatagtgattttcactctccagtaatccttgtagttgagcttcacatgccagtggattcttatcgggcacatgtgggaagtgcattgtacaaggggctgaccagtccatttgccagggccatcaattgcagcttgtagattaggtgaatcctcactgcaaaatgcaatagaattcatttaaaaactttagataaaaaatagaaccctaataggacatgaattaagagcaaaaggcagatcaactcacttcacacagtttgacccacttgggttctggcagccacatgagcatgttgggcagttcacaattgtgtcattataaaacgatgatagagatacacagcaggatggagtcttctgagcaagaaattgggaatatgtgcaggtcacattccatgtcactgcagataaagaatgtctctgttaagaaccctctctgctataaaatctagacaaaagtgcaacttgtatggaattcttcctaggactatctgcgattagaatatattattttgtgaagaacaaacaaaaaagaagaaaagagactgcattttttgttgatcctagtagtaacttattgtcagcaaatatcaattagcattaatcctttgtgaacaaattcctcttgttagattgtttccatttttactagcctggcaactaatgtaacctgaaaatttggaatcatggtcaaggaacaggaacacactaaataatatgatgtagctgtacctcacctccagaacatacataagcaactgatagcaagtaaaatgtaaaaattcagtacatgggcaacttacttagagcttgggttgccctgcgcccgtccgcggtgaaaaacttcgtaggcctgccaacaatggcacgcccacatgtgtacccagggcctggagtcttaagagtgaagttcctgggcaccttaacagttttattggtagttccagcaagaccaacactgatctggaaggaggaagcagcatttgctgggtcctggttaaaggtatttacaactcctgccttgcagcaattggcaatttg

SEQ ID NO: 21 - DNA, 1147 bp, Zea mays
tctgttgttgatcgacgctgggaagaaagaaagaaagaacacgatgtgcacgcacggatcagatcaggaagacggatggcgagagcgcaggacaagaattggccgtgcggggctacctgacgcattgtggcgacggtggggacccttggcagccgcagctgcaccgcgggcaatctacgatcgtctcgctgtagaaggtcgtcatggagacgcagcacgacggcgccgccgacgcccggtactgcgagtacgagcaggtcacctgccatgtcactgcacggagttcagctcgatcctctggtggcggtggtgcatatatatgcacgagaacgaacgcggcctgtctttagtgacgacgaccaaagagacaagaagaagaaaaaacgccttacggagcgcctggacgtagcggttcttgtcgaccttgatcctggtcggggccaccgtggtcgcgttgctgcaggtgtaccccggcacgcccatgtcgaactgccacggcttctcgggctccttgccgccgctgtccctggcgagggcgaactcgccgaccaccatctggaacgcggcggcggacgtcaggtcgctctggacgagagacgacagcacgccgccccggcagcagttggcgacctgcatgttgtacggcgtgccaggcggcaggtccaccatgacgggccgcttctggcagcaatgcgggcggctgcccccgctgccgacgcgggagcagtcgccctgctccgtcgtctccgcgcccgtcgtgctccagatgacctccttgccggcccagctccagctcagccgccaccccggacgctcgatgtgtcggtacatctggtagttgtggatgctcaccatgacctacgcacggagcaaatcgattgagatcttctctccttcgatcaggagacatgcttaatctccagacacacatgcgcgcttaatcatggaagggagaaagtgacgacttggaacgtgaaaacacacacacacacactcttcgtatcggtagcattaaccagtaaggacaggaagagatgaagtcagaatctttctgggtgtacatcagccggaagattcagtaagatggcgatatgctaaaactcacaagaaagcacgtatgcgcaccgtgtacggggtcatgcccgcg

SEQ ID NO: 22 - DNA, 769 bp, Zea mays
cgccgccgctcctgcccgcgcgcttcgtcgccgcctccgtcgcgctgctcgccgtcgccttctcctcctctctaacgcgtccgtcaggtcagaccagtgcgccgcgcgcacctccgcctccaaaccctgccatctcctgtcctcgtcggatgattcttgtgatgttcagatatatctccctcgtataatctcaatcacacataaaacaaagcttcctttcgtaccataccattaccatgaatgctgctgcatgaaacttttttttttttgcctgcaggtgcatacgatccgctcgatccgaacgggaacataacaatcaagtgggacgtgatacagtggactgcggatggctatgtggtgagtgaacgggttaattaattcgccactatctgacgacggacaccttctgatcgaaacgccctgcttcttcgttcccctcccctcccatgcccgtgcccaggccgtcgtttcgctatacaactaccagcagtaccgccacatccaggcgccgccggggtggaggctaggctgggtgtgggcgaagaaggaggtgatctgggcgatgaccggcggccaggccaccgagcagggcgactgctccaggttcaaggccagcgtcctcccccactgctgcaggagggacccggaggtggtggacctgctgcccgggactccctacaacacgcagaccgccaactgctgcaggggaggagtgctcgcctcgtgggcgcaggaccctagcgacgccgtcgcctcgttcaggtcagcg

SEQ ID NO: 23 - DNA, 725 bp, Zea mays
cggactgcacgttcccgtctggcccggccgtcatgagcagatcgttgtagtacttgatgccccatagcatcgccgtgtcgtctgcacgcgcgcggaaaaaaaaagagaaagaaaagattgaatttcttcagtgggggcgaacgaggtccaggaccaggtggtggtgctcgatctcactgatcactccgtaggggttgagaggtctgtagttgaagctgaaaatggtggtgaggttgtcgaagttggggtgctgcgcgaccaggttccactgcgagtagttcatccggtagttgaagttggtgaccgtgatcttcaccctccagtactccttgtagctgaccttgacgtgccagtgcacccttaccgggcacatgtgtgaggtgcactggactagcggcgccaagctgttcttgctaggatcgttgacgacggaagccagatagggcgaccttctactacccctgtccaaggacaggcaggcggacaacacgcatcgagtccagcagtattcacataactgaacatgatgaaaatggtgtgcgtgcgtgcgtgcgtgtgtgtgtttgtgtcgatcgaagctgagttcgatctgtggatgcaaattaaacttactctacgcagcttcctggcgcggcggtgctactgctgttgttgttctggcagccgcaggagcatgctgggcagctaacaatggtgtcgtttgtagacgacgagagcgagacaca

SEQ ID NO: 24 - DNA, 2048 bp, Zea mays
ataaagatggtggttgcgacgactacgaggaggacgagaagaagaagccgcagttcaaggcgcaggaggcgtgcaacggcgtgttcctgacgtacacgttcatggagcgcgccaaggagtacccgcacctgaagaaggcggcggcgcagccgtacgcgttcaaggccacggcgacggtgctcaacaccatgaccgaggacctcaaggcgtggcagatgttcgtgggcttccagcacaaggagatcctcgtgtccgtcggcggcgccgtgctgctcgacggctccgacctccccgccaacgtgtccggtggcgccacctttgcgggatacccaatggccgacctcctcaactccatcgagacggcgggcgagccgtccctgatcgagagcaagattgagatcaccggcacccaattcggcgtgaaggcccccgggaagcccatgcccaagaccatcaagttgaccaaccccgtgggcttccggtgccccgcccccaaccacaaaggtacgacgcgtcgtcatttcgccgccatgtctgtctgtggctgtgtggtatggcatgtcacgtcggccatggcctccaccaataacaaaaactgcaatgcaatgcaattgcagacagcgtgatgtacgtgtgctgcgtcaaggaccgcaagttcaaggcgaagaaggctaacagcacgcggtaccagacacggcggaaagcggacctgacgttcgcctacgacgtgctgcaggccaacaccaacaactaccaggtgcaggtgaccatcgacaactggagccccatcagccggctggacaactggaacctcacctgggagtggaagcgcggcgagttcatctacagcatgaagggcgcctacacgctgctcaaggaaggccccgcctgcatctacagccccgcagcgggctactacaaggacatggacttcacccccgtctacaactgcgagaagcggcccgtcatcgtggacctcccgccggagcgggagaaggacgacgccgtcgggaacctccccttctgctgcaagaacggcacgctgctgccgcccaccatggacccgtccaagtcgcgggccatgttccagatgcaggtgtacaagctgccgccggacctgaaccgcacggcgctgtacccgccgcagaactggaagatctccggcaagctcaacccgcagtacgcgtgcgggccgcccgtccgcgtgagcccccaggagttcccggacccgacgggtctcatgtcgaccacccccgccgtggcgtcgtggcaggtggcgtgcaacatcacgcggcccaagaagcgcgcctccaagtgctgcgtctccttctccgcctactacaacgactccgtggtgccgtgcaacacctgcgcctgcggctgcggcgacgacaccgcgacgtgcgacccggacaagcgcgccatgctgctgccaccggaggcgctgctcgtcccgttcgacaaccggtcggccaaggcacgggcgtgggccaagatcaagcactggcgggtgcccaaccccatgccgtgcagcgacaactgcggcgtcagcatcaactggcacgtcatcaacaactacaagtccggctggtcggcgcgcatgaccatcttcaactggcaggactacaccttcaaggattggtttgccgcagtgaccatgggcagccacttcagcggctacgagaacgtctactccttcaacggcacgcggatgggcgcccccttcaacaacaccatcttcatgcagggggtgccgggcctcgcttacctcgagcccatcaccgacgcgaagacgacatcggaacccaggcttcccggcaagcagcagtcggtcatctcgttcaccaggaaagacgcgcccaatgtcaacattcccagaggggaaggcttccccaagaggatctacttcgacggcgaggagtgcgcgctcccggataggatacccaaggtgtcgagcgcgcgccggcgggctgggaccgcgagcctgggtcagatagccatggcggcggcgctcgtgatgattgtggcgctagatggattcccttgtg

SEQ ID NO: 25 - DNA, 473 bp, Zea mays
cccgtcctgcagcagcatctcgttgtagtaacgtaacccccagaacatccccgtgtcgtctgcaagaaccaattgagcctcgcatcgcatcacagtagagtagacccgcgattatgctacagatttgtgctgcgggcatggtcacttactgtaggcgccgtactcgacgagaggcctgtagttgaagctgaacagctgcgtcaggctccgcaggttggggtgctgcagcaccaggttccagtcgctgtagttcctcgccaggttgtagttggacaccgtcaccttcacccgccagtacttgcggtagttcgtcttcacgtgccagtgcacccggatcgggcacatgtgctcggagcaccagacgatcggcgccgacgacggctcgtcgtcgccgacggccggcaaccatggttgttgttgatcgacgctggaagaaagaaagaaagaaacacgatgtgcacgcacggatcaga

SEQ ID NO: 26 - DNA, 847 bp, Zea mays
tggcacaagcagtgcctccggtggcagcagcatggattgtgcggtggtgctgcacgttggccctcgcctgtttgcagggcacccacaagcgcaggtgctgcaggggatcactgagtcgttgtagtacgccgagaaggtcacacaacacttgggcttggccccctttgtcgtggtaatgttgcacaccacctgccatgttgccacagcaagcgtcgtcgagtcaagcccgctcgggtctgggaacgcggttgggctgacaggcaccggctggccacaggcatagtccgggttcagcgatgatgcacccacgatcttgaaattagcaggggggaacagcttagtccggttcaggtctggtggcatcttgaaaacttgcatctggaacgcagatttcgactgtgcctcgtccatggacttgggcaagattgtcccattcctgcagcaattgtcaatcttcccaatctgagtgtcgttgtaccgggacaggggcaggtcaaggatcaccggcttgcggtcacaattgagcacctgcgaaaaatcaaggctctggtagtactgcccaggcgccccacagatacagcccgaggtgtccacctctgatgggtgagctcctttcattgagtagatgaactccccacgccgccactcccacgacagccgccagttgtcgaggcggccgagcttggcgttgttctcgagcgtgacgagcgcaaggtagctggaggggtaggcctggagcacatcgtaggtgatgacgaggtcgccggtgccgcgcggcaggaaatccttggtcgggtcggtggtgttggcgtcgatggcagtggcgttggcctcggcctccggcgtg

SEQ ID NO: 27 - DNA, 2074 bp, Zea mays
  misc_feature (786)..(786): n = a, c, g or t
ggaaagcagcgctgcggagcagagtgtgtcgcttcgctgtaaaaacaggggagagggagacgcgcccgctgccagtgcctgccgcacacgcgtttagcgtttaagttccactcctcgccgccccagatctccgccctcctcaccactgcccctcattccccggcgcccagcacccggcggccgcaaccgccgcagtccggagcaagatcggcgggtagacggacggacggacgggcgacaggcgggcgggcgcggctctgtctgtatctatctgttggtgggagaccggttgtgtcggttaggcggcggcgggtgggaaggaagaatggcggcgggcggcagatccatcgcgtgctttgccgccgtgctgctcgcggccgcgctgctcctcymcgcrycsrcyrcmacagaggcytaygattcgctggatccaaatggcaacatcaccataaaatgggatatcatgcagtggactcctgatggatatgtcgctgttgtcacaatgtttaattatcaacaatttcggcatatcggcgcacctggttggcagcttgggtggacatgggcaaagaaggaggttatatggtcaatggttggggctcagaccactgaacagggcgactgctcaaagttcaagagcagcccaccccattgctgcaagaaagatccaacaattgtcgatttacttccaggcactccatacaacatgcaaattgccaattgctgcaaggcaggagttgtaaatacctttaaccaggacccagcaaatgctgcttcctccttccnagatcnagtgnttggtcttgctnggaactaccaantaaaactgttaaggtngcccaggaacttcnactcttaagactccnaggccctgggtacnacatgntgggcgtgctattgttggcaggccaacgaagtttttcactgncggatgggcgcnagggtaacccaagctctaatgacnatggaatgtgacctgcacatattcccaatttcttgctcagaagactccrtcctgctgtgtatctctctcatcattttataatgacacaattgtgaactgcccgacatgctcatgtggctgccagaacccaagtgggtcaaactgtgtgaacgaggattcacctaatctacaagccgcaattgatggtcctggtaaatggactggccagcctcttgtacaatgcacttctcasatgtgcccaataagaatccactgggcatgtgaagctcaactacaaggaatactggagagtgaaaatcactatcacgaacttcaacttccgcatgaattacacacagtggaacttagttgctcagcatccaaactttgataatatcactcagttgttcagcttcaactacaaaccacttactccatatgggggtggcataaatgatacggcaatgttctggggtgtaaarttctacaatgatttgctgatgcaagccggcaaacttgggaatgtgcaatcagaactgcttctccgcaaggactcacggactttcachttcgaaaagggatgggccttcccacgccgagtgtacttcaatggtgataattgtgtcatgccatctcctgaaaattatccatggctgccgaatgcaagccctctaacaaaacaagcattgrcactcccaytcttgrtattctgggttgccttggctgytctgttggcttatgcatgatkagtgggatcaagakgtttagcaagyttcaagttgatgtcrgattccatgaggtgcactgcaacrrgwyatttrttcattcaattccatrgykgcacagraragatgaggcgawgccaagaaaaagtsgatgtgtrtgtstrtgtgtttgtaagttaaagggccaaaatgtatttcttgtytggtagtatatagcagcyctacaacactttggtgaacttagttactgcarattaggyaattacagttgcaccttttgtattttatagcaaacccagaayttytcattggattctaygactgcccctcttgtagtaaaygcaaggcttcmctgrtactcctgtttaaagattkgtsrawtrgrwgagacrayggtgattgwsat

SEQ ID NO: 28 - DNA, 1948 bp, Zea mays
  misc_feature (42)..(43): n = a, c, g or t
gcacgagssatcggmgctyskgctgctactgcyackmkwccnncgctagctagcagcagccgccggccggctcgcgcaagctaaggaagggtcgacatgacgatggggctccgcgtccgcgactcctccgcgctgctggctctggccgtcgcgctcgcctgctgctccgttgcagtggtggcctacgaccccctggacccgaacggcaacatcaccatcaagtgggacgtgatctcgtggacgcccgacgggtacgtggcgatggtgacgatgagcaactaccagatgtaccgggcacatcatggcgcccggntggacgttggggtggtcgtgggccaagaaggagggtgatctggtccatcgtgggsgcgcargccacggaagcaagggggactgctcccangtttcaaggggcgggcatcccgcactgctgcaagcncaaccccggccggtggtgggacytcctncccgnnngngcncyacaaccancagatcgccaactgctgcaaggccggcgtngtgtcgncgtncgggcarnayncggsnnnntccntctccgcgttccaargtctccgtcggcctskccggyaccaccaacaagacnntgaagctnnnmagraanttnacgctcatngggcccgggctgggctacacctgcnggcccgncgccgtggygccgtccaccgtgtactggacgcccgacnaccggcgccgnanncnnncgcctcatgacgtggacggtgacctgcacctactnckcaagcaagctggngtcccggtacccgtcytgctgcgtctccttctcctccttctacaaacaancaccaattcgttgccgtgccgcccggtgacgcgttgcgggctgnccggtntgnccangggmggsyamgcgggtccgggcggctgcatcgagggggactccaagcgcgcgctgtcggccggggtgaacacgccgcgcaaggacggccaggcgctgctgcagtgcacgccgcacatgtgccccatccgsgtscrctggcacgtcaagctcaactacaaggactactggcgcgccaagatcgccatcaccaactacaactacaggatgaactacacgcagtggacgctggtggcgcagcaccccaacctggacaacgtcaccgaggnnntcagcttccagtacaagccgctgcaaccatacgggagcatcagtgagtataatcatcgtcatctgatgacatgacatgacatgtacataatcatcggtgtctcaaatatatatatgcaattaatgcagatgacactggcatgtncnacgggnncaagtnnnacaacgacntnnncatggaggccggcccgttcggcaacgtgcagtcggaggtnnncatgcgcaagracgcaaggaccttcancttcagcatgggctgggcgttcccgcgcaagatytacttcaacggcgacgagtgcaagatgccgccgccggactcctnnnnctacctgcccaacgccgcgcccgttygtcgcntcgcannnggtcctgtccgccgccgcctsggsgttyctacwgttgnngytcctgntggcatgmccgtgmccgaaccaagggcaaggcctccgttttgttttcccgtytcgtcccgtggggcagggagcagayttcagtangcanggcattttatttggtttttttgcmaaggattcaacacttgggttttscgtcarnnnaaaactgtcgtgtatgtagtgtgagttgcangtcgtcsgatyccmacgtagtacaagmcaatytttggwtywanaatatgcaaaacgtgaatcarcmcnccaggatcwtsgyyycywmcaagannnrmagmaagraaaaaaawwmwmawrawrarwrawrwrwmamymgmsmyatakwtmtmtstgataatnmnnngtttcamcggttgcctgaacatttgctaacccatcagtagccactact

SEQ ID NO: 29 - DNA, 616 bp, Zea mays
  misc_feature (378)..(378): n = a, c, g or t
gcacgagagtgcatgcacgcccgatactgctagccaaggccaagccagtgcaggcgcggtggtgtgtgttgttctcgtcgcgcactcgccggcagcgatggagccccgccgctccgtgctgctcctggccctcgccgtcgccgccgcgctctccgtcgcagtggcttacgacccgttggacccgaacgggaacattaccatcaagtgggacatcatgtcgtggacgcccgacggctatgtcgcggtggtgaccatcaacaacttccagacgtaccggcagatcacggcgccggggtggacggtggggtggacgtgggcgaagcgggaggtgatctggtccatggtgggcgcgcaggccacggagcarggcgactgctncccgcttcnaaggccaacatcccgncactngctgcaagcgcaccccggccgtcgtcgacctgctccccggcgtgccctacaaccagcagatcgccaactgctgccgcggcggcgtcgtcagcgcctacggccaggacccggccaccgccgtcgccgcgttccaggtcagcgtcggccaggccggcaccaccaaccgcaccgtcaaggtgcccaagaacttccnnngctnnggnnnnng

SEQ ID NO: 30 - DNA, 550 bp, Zea mays
ccacgcgccggtcttcagcttcggctacaagcccgtcgtctcctatggatccatcaatgacacggccatgttctacgggctcaagtacttcaacgaccacctgatgcaggcggggccgtacgggaacgtgcagtcggaggtgctcatgcgcaaggacgccagcaccttcaccttcaggcagggctgggccttcccgcgcaaggtctacttcaacggcgacgagtgccagatgccgccgccggacgcctacccctacttgcccaactccgcgccgccgacagccgcggcgtcgctaggcggcgcagcggcagsggccgtcgtggtgctcttgggcatgatcgtggcatgagaaaacacgggacatcgatcgacctagtgctaggaccggcacaggggaatggaaaaaagacgttgctttcttctgtagatagagagaccagagacctcggtttgggtttcaggaatggtttggaactttggatgtttttctttcagtgtagatggacaagccatgattttgcaaggaaaattaacatgtgcatctctcgtc

SEQ ID NO: 31 - DNA, 19 bp, Artificial
cactccatacaacatgcaa

SEQ ID NO: 32 - DNA, 19 bp, Artificial
catttaccaggaccatcaa

SEQ ID NO: 33 - DNA, 19 bp, Artificial
aaccatacgggagcatcag

SEQ ID NO: 34 - DNA, 19 bp, Artificial
aaatgccctgcctactgaa

SEQ ID NO: 35 - DNA, 18 bp, Artificial
cgaacgggaacattacca

SEQ ID NO: 36 - DNA, 19 bp, Artificial
aagttcttgggcaccttga

SEQ ID NO: 37 - DNA, 19 bp, Artificial
ttgcggaagttgaagtttg

SEQ ID NO: 38 - DNA, 18 bp, Artificial
atggaatgtgacctgcac

SEQ ID NO: 39 - DNA, 19 bp, Artificial
tgacacggccatgttctac

SEQ ID NO: 40 - DNA, 19 bp, Artificial
aacccaaaccgaggtctct

SEQ ID NO: 41 - DNA, 40 bp, Artificial
aattaaccctcactaaagggcatacgggagcatcagtgag

SEQ ID NO: 42 - DNA, 43 bp, Artificial
gtaatacgactcactatagggcgacgacctgcaactcacacta

SEQ ID NO: 43 - DNA, 20 bp, Artificial
tcgacaacgagcaactcatc

SEQ ID NO: 44 - DNA, 20 bp, Artificial
ctgcagatggactggagtca

SEQ ID NO: 45 - DNA, 18 bp, Artificial
aaagcaaccgattgatgc

SEQ ID NO: 46 - DNA, 18 bp, Artificial
tccgacttccgagtgaga

SEQ ID NO: 47 - DNA, 28 bp, Artificial
gagacccaaccaaaactaataatctctt

SEQ ID NO: 48 - DNA, 24 bp, Artificial
ctgctgcagaccatttgaaataac

SEQ ID NO: 49 - DNA, 24 bp, Artificial
tggctgacgaactattttcattca

SEQ ID NO: 50 - DNA, 24 bp, Artificial
gattgctcagttcatgagggagat

SEQ ID NO: 51 - DNA, 39 bp, Artificial
aattaaccctcactaaagggccctacaaccagcagatcg

SEQ ID NO: 52 - DNA, 41 bp, Artificial
gtaatacgactcactatagggctgccagtgtcatctgcatt

SEQ ID NO: 53 - DNA, 18 bp, Artificial
agggagcttgtgctgcta

SEQ ID NO: 54 - DNA, 19 bp, Artificial
gcagcttcaccgtcttgtt

SEQ ID NO: 55 - DNA, 26 bp, Artificial
caagctaaggaagggtcgacatgacg

SEQ ID NO: 56 - DNA, 26 bp, Artificial
cggcttgtactggaagctgaagacct

SEQ ID NO: 57 - DNA, 32 bp, Artificial
agagaagccaacgccawcgcctcyatttcgtc

SEQ ID NO: 58 - DNA, 1797 bp, Zea mays
ccacgcgtccggggatcggagcttgtgctgctactgctactataccagcgctagctagcagcagccgccggccggctcgcgcaagctaaggaagggtcgacatgacgatggggctccgcgtccgcgactcctccgcgctgctggctctggccgtcgcgctcgcctgctgctccgttgcagtggtggcctacgaccccctggacccgaacggcaacatcaccatcaagtgggacgtgatctcgtggacgcccgacgggtacgtggcgatggtgacgatgagcaactaccagatgtaccggcacatcatggcgcccgggtggacgttggggtggtcgtgggccaagaaggaggtgatctggtccatcgtgggggcgcaggccacggagcagggggactgctccaagttcaagggcggcatcccgcactgctgcaagcgcaccccggccgtggtggacctcctcccgggggtgccctacaaccagcagatcgccaactgctgcaaggccggcgtggtgtcggcgtacgggcaggacccggcggggtccgtctccgcgttccaggtctccgtcggcctggccggtaccaccaacaagacggtgaagctgcccaggaacttcacgctcatggggcccgggctgggctacacctgcgggcccgccgccgtggtgccgtccaccgtgtactggacgcccgaccaccggcgccggacgcaggcgctcatgacgtggacggtgacctgcacctactcgcagcagctggcgtcccggtacccgtcctgctgcgtctccttctcctccttctacaacagcaccatcgtgccgtgcgcccggtgcgcgtgcggctgcggcggccacggcggccacgcgggtccgggcggctgcatcgagggggactccaagcgcgcgctgtcggccggggtgaacacgccgcgcaaggacggccaggcgctgctgcagtgcacgccgcacatgtgccccatccgggtgcactggcacgtcaagctcaactacaaggactactggcgcgccaagatcgccatcaccaactacaactacaggatgaactacacgcagtggacgctggtggcgcagcaccccaacctggacaacgtcaccgaggtcttcagcttccagtacaagccgctgcaaccatacgggagcatcaatgacactggcatgttctacgggctcaagttctacaacgactttctcatggaggccggcccgttcggcaacgtgcagtcggaggtgctcatgcgcaaggacgcaaggaccttcaccttcagcatgggctgggcgttcccgcgcaagatctacttcaacggcgacgagtgcaagatgccgccgccggactcctacccctacctgcccaacgccgcgcccgtcgtcgcctcgcagctggtcctgtccgccgccgcctcggcgttcctactgttgctgctcctggtggcatgaccgtgaccgaaccaagggcaaggcctccgttttgttttcccgtctcgtcccgtgggcagggagcagacttcagtaggcagggcattttatttggtttttttgccaaggattcaacacttgggttttcgtcagaggaaaactgtcgtgtatgtagtgtgagttgcaggtcgtcggatccccacgtacaagacaatctttggatctagaatatgcaaaacgtgaatcagcacgccaggatcatcgtctcctacaagattggcagaaaaaaaatctcatgatgagtgatgtgtcaacagacctatatatatgtgataatcactggtttcaaaaaaaaaaaaaaaaa

SEQ ID NO: 59 - protein, 448 aa, Zea mays
MGLRVRDSSALLALAVALACCSVAVVAYDPLDPNGNITIKWDVISWTPDGYVAMVTMSNYQMYRHIMAPGWTLGWSWAKKEVIWSIVGAQATEQGDCSKFKGGIPHCCKRTPAVVDLLPGVPYNQQIANCCKAGVVSAYGQDPAGSVSAFQVSVGLAGTTNKTVKLPRNFTLMGPGLGYTCGPAAVVPSTVYWTPDHRRRTQALMTWTVTCTYSQQLASRYPSCCVSFSSFYNSTIVPCARCACGCGGHGGHAGPGGCIEGDSKRALSAGVNTPRKDGQALLQCTPHMCPIRVHWHVKLNYKDYWRAKIAITNYNYRMNYTQWTLVAQHPNLDNVTEVFSFQYKPLQPYGSINDTGMFYGLKFYNDFLMEAGPFGNVQSEVLMRKDARTFTFSMGWAFPRKIYFNGDECKMPPPDSYPYLPNAAPVVASQLVLSAAASAFLLLLLLVA

SEQ ID NO: 60 - DNA, 1094 bp, Zea mays
tagtcctgtaagtttgggccgtgcctgctgggccagcacgagcccggcacgaaattaatggcacgaagcccggcccagcacgatcaaaaaatactcgggccagcacggcacgttaaacgggctgggccgtgctccggctttcggcccgacggcccaaatagcccggcacgccatagtgggccgtgctcgggccagcccggcacgatttagggttagggtttatttcccacacagcagtcacgcggtcacatctcacgcgccgccgctcgctcattttattcaccctcacgctgcggctctcgcggtctcgctctcgctggctcctcggttccttcgtcaatcgtccgtccgccgtcctcctcggtcctccctccggtcctccgccggcgactgttcggttccccgtgactctgtgcacttcctcggatttggaatggagtcatggatctgcgtctcatcggtaactctgcgactcctcgcctccagccctccaccaccatggccggatgcccgaagcttttatttgtttcgaaaatcgaaaccctaatcatgcttttttgctggaatttctagccttccacctcccaggaatcaatgcgccacgccgccaactcgccaccaccgcgagtccgcgagttcaacggttcaaccggctccactgctccagcaaggtattgttcatgtaaacattttccccactgtaatatggactgttattgttcatgtgtgctgttattgttcatgtgtaatatgggctgttattgttcatgtaaacattttccccactgttgtttatttaaatttatctagttcatgtgtgctgttgtttttgttgcatgagagatttgaacttgtttatgtatcggatctggtcatatgatgattaattgcgggccgggcctgggccagcacggcccgatgaaagcccgtcgtgctttagggccgtgctgggcctatattttaggagatgagcacgatttagcccggcccgaaagaaattcgtgctagcacggcccgaagcatctaagcccgaagcacgacgggcccgtgccgggccagcccggcccggcccaacttgcaggactagtggtggc

SEQ ID NO: 61 - DNA, 1798 bp, Zea mays
  misc_feature (668)..(668): n == a, c, g or t
ttgtgctgctactgctactataccagcgctagctagcagcagccgccggcctgctcgcgcaagctaaggaaaggtcgacatgacgatggggctccgcgtccgcgactcctccgcgctgctggctctggccgtcgcgctcgcctgctgctccgttgcaggttcggttaccatatttcattcatctgaaaatgtaaacagtgtcgatcattcgatgggcgacgctcaccttctctcctctcctgtcgccatggctggcggctgctgcacacactggcacttgcgcagtggtggcctacgaccccctggacccgaacggcaacatcaccatcaagtgggacgtgatctcgtggacgcccgacgggtacgtggcgatggtgacgatgagcaactaccagatgtaccggcacatcatggcgcccgggtggacgttggggtggtcgtgggccaagaaggaggtgatctggtccatcgtgggcgcgcaggccacggagcagggggactgctccaagttcaagggcggcatcccgcactgctgcaagcgcaccccggccgtggtggacctcctcccgggggtgccctacaaccagcagatcgccaactgctgcaaggccggcgtggtgtcggcgtacgggcaggacccggcggggtccgtctccgcgttccaggtntccgtcggcctggccggcaccaccaacaagacggtgaagctgcccaggaacttcacgctcatggggcccgggctgggctacacctgcgggcccgccgccgtggtgccgtccaccgtgtactggacgcccgaccaccggcgccggacgcaggcgctcatgacgtggacggtgacctgcacctactcgcagcagctggcgtcccggtacccgtcctgctgcgtctccttctcctccttctacaacagcaccatcgtgccgtgcgcccggtgcgcgtgcggctgcggcggccacggcggccacgcgggtccgggcggctgcatcgagggggactccaagcgcgcgctgtcggccggggtgaacacgccgcgcaaggacggccaggcgctgctgcagtgcacgccgcacatgtgccccatccgggtgcactggcacgtcaagctcaactacaaggactactggcgcgccaagatcgccatcaccaactacaactacaggatgaactacacgcagtggacgctggtggcgcagcaccccaacctggacaacgtcaccgaggtcttcagcttccagtacaagccgctgcaaccatacgggagcatcagtgagtataatcatcgtcatctgatgacatgacataacatgtacataatcatcggtctctcaaatatatattatgcaattaatgcagatgacactggcatgttctacgggctcaagttctacaacgactttctcatggaggccggcccgttcggcaacgtgcagtcggaggtgctcatgcgcaaggacgcaaggaccttcaccttcagcatgggctgggcgttcccgcgcaagatctacttcaacggcgacgagtgcaagatgccgccgccggactcctacccctacctgcccaacgccgcgcccgtcgtcgcctcgcagctggtcctgtccgccgccgcctcggcgtttctactgttgctgctcctggtggcatgaccgtgaccgaaccaagggcaaggcctccgttttgttttcccgtctcgtcccgtgggcagggagcagacttcagtaggcagggcattttatttggttttgccaaggattcaacacttgggttttcgtcagaggaaaactgtcgtgtatgtagt